SEQUENCE LISTING (114 sequences)

SEQ ID NO: 1 - protein, 16 aa, Bos primigenius
EQLQDMGLEDLFSPEK

SEQ ID NO: 2 - protein, 11 aa, Bos primigenius
TSDQIHFFFAK

SEQ ID NO: 3 - protein, 9 aa, Bos primigenius
FRIESFSVK

SEQ ID NO: 4 - protein, 21 aa, Bos primigenius
NNNDNIFLSPLSISTAFAMTK

SEQ ID NO: 5 - protein, 11 aa, Bos primigenius
SRLPGIVAEGR

SEQ ID NO: 6 - protein, 9 aa, Bos primigenius
SSELVSANR

SEQ ID NO: 7 - protein, 23 aa, Bos primigenius
AFLEVNEEGSEAAASTVISIAGR

SEQ ID NO: 8 - protein, 7 aa, Bos primigenius
LQPLDFK

SEQ ID NO: 9 - protein, 9 aa, Bos primigenius
LPGIVAEGR

SEQ ID NO: 10 - protein, 18 aa, Bos primigenius
KATEGQGSEQKIPGATNR

SEQ ID NO: 11 - protein, 7 aa, Bos primigenius
RVWELSK

SEQ ID NO: 12 - protein, 24 aa, Bos primigenius
ITDVIPPQAINEFTVLVLVNTIFK

SEQ ID NO: 13 - protein, 9 aa, Bos primigenius
SKFSPENTR

SEQ ID NO: 14 - protein, 28 aa, Bos primigenius
VEQELTPDMLQEWLDELTETLLVVHMPR

SEQ ID NO: 15 - protein, 7 aa, Bos primigenius
FDTISEK

SEQ ID NO: 16 - protein, 17 aa, Bos primigenius
ATEGQGSEQKIPGATNR

SEQ ID NO: 17 - protein, 11 aa, Leishmania mexicana
HLVPQALQLHR

SEQ ID NO: 18 - protein, 12 aa, Leishmania mexicana
VKLSSVSDAFQK

SEQ ID NO: 19 - protein, 9 aa, Leishmania mexicana
CIDGAFTPK

SEQ ID NO: 20 - protein, 7 aa, Leishmania mexicana
YDQLVTR

SEQ ID NO: 21 - protein, 10 aa, Leishmania mexicana
LSSVSDAFEK

SEQ ID NO: 22 - protein, 18 aa, Leishmania mexicana
GGYVTCPPYVEVCQGNVK

SEQ ID NO: 23 - protein, 10 aa, Leishmania mexicana
NVGCAFLSEK

SEQ ID NO: 24 - protein, 7 aa, Leishmania mexicana
TYSVQVR

SEQ ID NO: 25 - protein, 8 aa, Leishmania mexicana
AEEMPWGR

SEQ ID NO: 26 - protein, 8 aa, Leishmania mexicana
AANWGALR

SEQ ID NO: 27 - protein, 12 aa, Leishmania amazonensis
ADLVNNLGTIAR

SEQ ID NO: 28 - protein, 9 aa, Leishmania amazonensis
EVLGDKVEK

SEQ ID NO: 29 - protein, 13 aa, Leishmania amazonensis
EGVHFEESEEEKR

SEQ ID NO: 30 - protein, 15 aa, Leishmania amazonensis
TTEKEVTDEDEEEAK

SEQ ID NO: 31 - protein, 21 aa, Leishmania amazonensis
KADEDGEEPKVEEVTEGEEGK

SEQ ID NO: 32 - protein, 14 aa, Leishmania amazonensis
GVVDSEDLPLNISR

SEQ ID NO: 33 - protein, 12 aa, Leishmania amazonensis
VEEVTEGEEGKK

SEQ ID NO: 34 - protein, 13 aa, Leishmania amazonensis
TLTVEDNGIGMTK

SEQ ID NO: 35 - protein, 15 aa, Leishmania amazonensis
HSEFIGYDIELMVEK

SEQ ID NO: 36 - protein, 12 aa, Leishmania amazonensis
TMKEVLGDKVEK

SEQ ID NO: 37 - protein, 15 aa, Bos taurus
SVLGDVGITEVFSDR

SEQ ID NO: 38 - protein, 11 aa, Bos taurus
YASSANLHLPK

SEQ ID NO: 39 - protein, 9 aa, Bos taurus
GNTHTEILK

SEQ ID NO: 40 - protein, 10 aa, Bos taurus
LVDTFLEDVK

SEQ ID NO: 41 - protein, 17 aa, Bos taurus
VLDPNTVFALVNYISFK

SEQ ID NO: 42 - protein, 8 aa, Bos taurus
KINDYVEK

SEQ ID NO: 43 - protein, 11 aa, Bos taurus
DFHVDEQTTVK

SEQ ID NO: 44 - protein, 15 aa, Bos taurus
LQQLEDKLNNELLAK

SEQ ID NO: 45 - protein, 10 aa, Bos taurus
LSISETYDLK

SEQ ID NO: 46 - protein, 8 aa, Bos taurus
ADLSGITK

SEQ ID NO: 47 - protein, 7 aa, Bos taurus
INDYVEK

SEQ ID NO: 48 - protein, 9 aa, Leishmania donovani
EIVDLALDR

SEQ ID NO: 49 - protein, 8 aa, Leishmania donovani
LSVDYGKK

SEQ ID NO: 50 - protein, 14 aa, Leishmania donovani
SLDIERPSYTNVNR

SEQ ID NO: 51 - protein, 21 aa, Leishmania donovani
QLFNPEQLVSGKEDAANNYAR

SEQ ID NO: 52 - protein, 7 aa, Leishmania donovani
GHYTIGK

SEQ ID NO: 53 - protein, 12 aa, Leishmania amazonensis
INVYFDESAGGR

SEQ ID NO: 54 - protein, 10 aa, Leishmania amazonensis
LAVNLVPFPR

SEQ ID NO: 55 - protein, 10 aa, Leishmania amazonensis
VGEQFTGMFR

SEQ ID NO: 56 - protein, 15 aa, Leishmania amazonensis
AVLMDLEPGTMDSVR

SEQ ID NO: 57 - protein, 10 aa, Leishmania donovani
STATGHLIYK

SEQ ID NO: 58 - protein, 7 aa, Leishmania donovani
VGYNPEK

SEQ ID NO: 59 - protein, 7 aa, Leishmania donovani
EAAEIGK

SEQ ID NO: 60 - protein, 16 aa, Leishmania donovani
SVFTIIDAPGHRDFIK

SEQ ID NO: 61 - protein, 6 aa, Leishmania donovani
YDEISK

SEQ ID NO: 62 - protein, 29 aa, Leishmania donovani
NMITGTSQADAAILMIDSTHGGFEAGISK

SEQ ID NO: 63 - protein, 14 aa, Leishmania infantum
DYGVLIEESGIALR

SEQ ID NO: 64 - protein, 7 aa, Leishmania infantum
NVDEALR

SEQ ID NO: 65 - protein, 17 aa, Leishmania infantum
EAAPQFSGQAVVNGAIK

SEQ ID NO: 66 - protein, 16 aa, Leishmania infantum
KKGGLGEMHIPVLADK

SEQ ID NO: 67 - protein, 14 aa, Leishmania infantum
GGLGEMHIPVLADK

SEQ ID NO: 68 - protein, 8 aa, Leishmania infantum
GLFIIDKK

SEQ ID NO: 69 - protein, 14 aa, Leishmania tarentolae
YTGSVIALYPEFVK

SEQ ID NO: 70 - protein, 11 aa, Leishmania tarentolae
SSLTQEVMSER

SEQ ID NO: 71 - protein, 17 aa, Leishmania tarentolae
FQTGGPDGDLGSNEVLR

SEQ ID NO: 72 - protein, 12 aa, Leishmania tarentolae
LPDGTLVEDGSR

SEQ ID NO: 73 - protein, 17 aa, Leishmania mexicana
ESAAPGTSTSSETPAPR

SEQ ID NO: 74 - protein, 17 aa, Leishmania mexicana
NGPIAIALDASSFMSYK

SEQ ID NO: 75 - protein, 11 aa, Leishmania mexicana
AYETLAEEQQR

SEQ ID NO: 76 - protein, 10 aa, Leishmania mexicana
NPHAQFGITK

SEQ ID NO: 77 - protein, 21 aa, Bos taurus
IVSGISEGQVETVQSSPVTFR

SEQ ID NO: 78 - protein, 10 aa, Bos taurus
ALETHTYFER

SEQ ID NO: 79 - protein, 9 aa, Bos taurus
FWTFPEVLR

SEQ ID NO: 80 - protein, 16 aa, Bos taurus
DVAMKPPSVYLLPPTR

SEQ ID NO: 81 - protein, 11 aa, Bos taurus
HLVDEPQNLIK

SEQ ID NO: 82 - protein, 16 aa, Bos taurus
DVAMKPPSVYLLPPTR

SEQ ID NO: 83 - protein, 10 aa, Bos taurus
LVNELTEFAK

SEQ ID NO: 84 - protein, 8 aa, Bos taurus
AEFVEVTK

SEQ ID NO: 85 - protein, 10 aa, Bos taurus
HTLNQIDSVK

SEQ ID NO: 86 - protein, 11 aa, Bos taurus
QDGQFSVLFTK

SEQ ID NO: 87 - protein, 8 aa, Bos taurus
ALGGEDVR

SEQ ID NO: 88 - protein, 7 aa, Bos taurus
EVVDPTK

SEQ ID NO: 89 - protein, 10 aa, Leishmania chagasi
LLEAFQFVEK

SEQ ID NO: 90 - protein, 11 aa, Leishmania chagasi
QITVNDMPVGR

SEQ ID NO: 91 - protein, 7 aa, Leishmania chagasi
NVEEVLR

SEQ ID NO: 92 - protein, 16 aa, Leishmania major
VVNEPTAAALAYGMDK

SEQ ID NO: 93 - protein, 13 aa, Leishmania major
EINDVVLVGGMTR

SEQ ID NO: 94 - protein, 18 aa, Leishmania major
SQVLLTYIDGDQYRPVSR

SEQ ID NO: 95 - protein, 10 aa, Leishmania major
TAAGIASYMK

SEQ ID NO: 96 - protein, 13 aa, Dictyostelium discoideum
IQDKEGIPPDQQR

SEQ ID NO: 97 - protein, 9 aa, Dictyostelium discoideum
TLSDYNIQK

SEQ ID NO: 98 - protein, 16 aa, Leishmania amazonensis
LEETAAELDALLQEER

SEQ ID NO: 99 - protein, 17 aa, Leishmania amazonensis
RLEETAAELDALLQEER

SEQ ID NO: 100 - protein, 16 aa, Leishmania mexicana
APAVPEGTPVVNALNR

SEQ ID NO: 101 - protein, 11 aa, Leishmania mexicana
SNSVLFPEGAR

SEQ ID NO: 102 - protein, 17 aa, Leishmania amazonensis
IAVSAEDLTDPAYHCAR

SEQ ID NO: 103 - protein, 8 aa, Leishmania amazonensis
AEAMPWGR

SEQ ID NO: 104 - protein, 19 aa, Leishmania major
SALVGDATDLFDFIAQSVR

SEQ ID NO: 105 - protein, 14 aa, Trypanosoma brucei
FTQANSEVSALLGR

SEQ ID NO: 106 - protein, 19 aa, Leishmania pifanoi
YQRPLSYTDLFALSATADR

SEQ ID NO: 107 - protein, 14 aa, Homo sapiens
SLNNKFASFIDKVR

SEQ ID NO: 108 - protein, 13 aa, Lupinus albus
TLEAEAAHGTVTR

SEQ ID NO: 109 - protein, 9 aa, Leishmania major
AELEAQVAR

SEQ ID NO: 110 - protein, 14 aa, Drosophila melanogaster
SGQVVPGYGHAVLR

SEQ ID NO: 111 - protein, 13 aa, Leishmania mexicana
HVSGDHLGGHAVK

SEQ ID NO: 112 - protein, 24 aa, Unknown
  Probable eukaryotic initiation factor 4A (eIF4A) (eIF-4A) gi I643070I gb IAAA80219.1I ribosomal DEAD box protein.
VLVLDEADEMLSQGFADQIYEIFR

SEQ ID NO: 113 - protein, 9 aa, Bos taurus
GEDLANINR

SEQ ID NO: 114 - protein, 12 aa, Bos taurus
LYDQLLQSYQQK

(19) United States
(12) Patent Application Publication
(10) Pub. No.: US 2014/0356391 A1
(43) Pub. Date: Dec. 4, 2014

(54) AMINO ACID SEQUENCES FOR CLINICAL REMISSION OF PSORIASIS AND RELATED DISEASES.

(71) Applicant: Jose Antonio O'Daly, Irvington, NJ (US)

(72) Inventor: Jose Antonio O'Daly, Irvington, NJ (US)

(21) Appl. No.: 13/904,440

(22) Filed: May 29, 2013

(51) Int. Cl. A61K 39/008

(52) U.S. Cl. CPC A61K 39/008; A61K 2039/55505
USPC 424/191.1

(57) ABSTRACT

Polypeptides comprising amino acid sequences of particulate antigens isolated from various species of Leishmania protozoa, or immunogenic variants thereof, are disclosed for the treatment and clinical remission of cutaneous leishmaniasis, psoriasis, psoriatic arthritis, rheumatoid arthritis, atopic dermatitis, seborrheic dermatitis and skin papilloma. Also disclosed are nucleic acid sequences encoding such polypeptides, vectors incorporating such nucleic acid sequences, methods for genetically engineering microbial host cells to produce such polypeptides, and such recombinant microbial host cells. The polypeptides induced a TH1 cellular immune response, a positive intradermic reaction, and a blastogenic response in peripheral blood lymphocytes after clinical remission of lesions. Populations of peripheral blood lymphocytes that are altered in psoriasis and psoriatic arthritis patients returned to normal values in patients who received the polypeptides and experienced clinical remission of lesions after treatment.

### REFERENCES CITED

U.S. PATENT DOCUMENTS

| | | |
|---|---|---|
| 4,687,666 A | August 1987 | O'Daly |
| 6,673,351 B1 | January 2004 | O'Daly |
| 4,992,273 A | February 1991 | Monjour et al. |
| 5,202,320 A | April 1993 | Tidwell et al. |
| 5,411,865 A | May 1995 | Redd |
| 5,496,830 A | March 1996 | Shapiro et al. |
| 5,541,196 A | July 1996 | Fournet et al. |
| 5,658,952 A | August 1997 | Jacobus |
| 5,674, 503 A | October 1997 | Olafson |
| 5,719,263 A | February 1998 | Reed |
| 5,726,292 A | March 1998 | Lowell |
| 5,733,778 A | July 1998 | Matlashewski et al. |
| 5,827,671 A | October 1998 | Matlashewski et al. |
| 5,834,592 A | November 1998 | Reed et al. |
| 5,876,735 A | March 1999 | Reed |
| 5,912,166 A | June 1999 | Reed et al. |
| 5,965,142 A | October 1999 | Dillion et al. |
| 6,013,268 A | January 2000 | Reed |
| 6,027,934 A | February 2000 | Powell |
| 6,048,530 A | April 2000 | Srivastava |
| 6,057,142 A | May 2000 | Aerts |
| 6,063,902 A | May 2000 | Stiegler |
| 6,087,341 A | July 2000 | Khavari et al. |
| 6,133,017 A | October 2000 | Matlashewski et al. |
| 6,139,844 A | October 2000 | Alkemade et al. |
| 6,162,612 A | December 2000 | Giordano |
| 6,162,638 A | December 2000 | Papadopoulu et al. |
| 6,165,735 A | December 2000 | Chandrashekar et al. |
| 6,168,923 B1 | January 2001 | Scott et al. |
| 6,174,995 B1 | January 2001 | Li et al. |

FOREIGN PATENT DOCUMENTS

| | | |
|---|---|---|
| EP | 0135108 | March 1985 |
| EP | 0276783 A2 | August 1988 |
| EP | 0293827 A2 | December 1988 |
| EP | 0703295 A1 | March 1996 |
| EP | 0806476 A2 | November 1997 |
| FR | 2570947 A1 | April 1986 |
| FR | 2612779 A1 | September 1988 |
| FR | 2615103 A1 | November 1998 |
| WO | WO 89/01045 | February 1989 |
| WO | WO 95/06628 | March 1995 |
| WO | WO 95/30006 | November 1995 |
| WO | WO 00/39298 | July 2000 |

### OTHER PUBLICATIONS

O'Daly, J A et al. 1995, Proteinas de amastigotes de varias cepas de leishmanias protegen a seres humanos contra la Leishmaniasis en el área de Guatire, Edo. Miranda, Venezuela. Gaceta Médica de Caracas. 103:133-177.
O'Daly, J A et al. 1995. Comparación de los efectos terapéuticos de la mezcla de promastigotes+BCG, antigenos purificados de amastigotes y el Glucantime en un área hiperendémica de Leishmaniasis cutánea, en Guatire, Edo, Miranda, Venezuela. Gaceta Médica de Caracas. Gaceta Médica de Caracas 103:327-357.
O'Daly, J A et al. 2013, Clinical and immunological analysis of cutaneous leishmaniasis, before and after different treatments. Journal Parasitology Research. Volume 2013. dx/doi.org/10.1155/2013/657016.
O'Daly, J A et al. 2009, Antigens from Leishmania amastigotes induced clinical remission of psoriasis. Arch. Dermatol. Res. 301:1-13.
O'Daly, J A et al. 2010, Lymphocyte subsets in peripheral blood of patients with psoriasis before and after treatment with leishmania antigens. Arch. Dermatol. Res. 302, 95-104.
O'Daly, J A et al. 2011, Antigens from Leishmania amastigotes inducing clinical remission of psoriatic arthritis. Arch. Dermatol. Res. 303:399-415.
O'Daly J A 2011, Psoriatic Disease: A Systemic Pathology, Structured by Psoriasis, Psoriatic Arthritis and Comorbidities. J. Clin. Rheumatol. Musculoskel. Med. 2:1-11.
O'Daly J A et al. 2009, Isolation of Leishmania amastigote protein fractions which induced lymphocyte stimulation and remission of psoriasis. Arch. Dermatol. Res. 301:411-427.
O'Daly, J. A. et al. 2010, Purified proteins from Leishmania amastigotes -induced delayed type hypersensitivity reactions and remission of collagen-induced arthritis in animal models. Arch Dermatol Res. 302:567-581.
O'Daly J A Psoriasis, a Systemic Disease Beyond the Skin, as Evidenced by Psoriatic Arthritis and Many Comorbidities—Clinical Remission with a Leishmania Amastigotes Vaccine, a Serendipity Finding 2012 pp 1-56, InTech publishers. Croatia.
O'Daly J A and Rodriguez M B, 1988, Differential growth requirement of several Leishmania spp in chemically defined culture media. Acta Trop. 45:109-126. Bretaña A and O'Daly J A 1976, Uptake of fetal proteins by Trypanosoma cruzi immunofluorescence and ultrastructural studies. Internat. J. for Parasitol. 6:379-386
O'Daly J A and Cabrera Z, 1986, Immunization of hamsters with TLCK-killed parasites induces protection against Leishmania infection. Acta Trop 43:225-36. lesions after treatment.

### FIELD OF THE INVENTION

The present invention relates generally to vaccines or immunotherapeutic agents, compositions comprising those agents, and methods of use of those agents and compositions for the treatment and clinical remission of cutaneous leishmaniasis, psoriasis, psoriatic arthritis, rheumatoid arthritis, atopic dermatitis, seborrheic dermatitis and skin papilloma.

### BACKGROUND

Psoriasis is a systemic, chronic, relapsing inflammatory skin disorder, affecting 1-3% of the world inhabitants. It has wide clinical spectra that ranges from epidermal (scaly) and vascular (thickened erythematous) involvements, to the malignant form known as generalized erythrodermia (Christophers and Mrowietz, 2003, Psoriasis. Fitzpatrick's Dermatology in General Medicine, 6th edn. McGraw-Hill, New York; pp 407-427). It is genetically determined by multiple genes interacting with environmental factors (Farber and Nall, 1974, Dermatologica 148:1-18. Gudjonsson et al. 2004, Clin. Exp. Immunol. 135:1-82).

The inflammatory process is immune mediated by unknown antigens through binding, activation and co-stimulation of T cells by, antigen presenting cells (APC), dendritic dermal and epidermal cells (DC), Langerhans cells (LC) and macrophages in epidermis and dermis. A multimolecular complex is formed, between APC and T cells: the immunological synapse structured by major histocompatibility complex (MHC) and T cell receptors (TCR). At the beginning of lesion formation APC travel from the skin to the lymph nodes and after activation, CD4+ T cells proliferate and migrate from the lymph nodes to the skin, mainly in the dermis by a process regulated by a cytokine network, and cell-cell interactions involving, naive T cells, memory T cells expressing cutaneous lymphocyte antigen (CLA), the endothelium receptors intercellular adhesion molecule (ICAM)-1 and E-Selectin binding dermal and papillary endothelial cells. Also in the epidermis are neutrophiles and CD8+ T cells expressing MHC-I, and interleukin (IL)-2 receptors (Wang et al. 2004, G. Ital. Dermatol. Venereol. 139:207-230. Santamaria-Babi et al. 1995, J. Immunol. 154:1543-1550).

In newer lesions granulocytes are in the epidermis with lymphocytes, monocytes and macrophages forming a psoriatic hallmark: sterile spongiform micro-abscesses (Munro abscesses), increasing with disease activity. Physical trauma to the skin, results in a psoriatic lesion (Koebner phenomenon), which increases when the disease is active (Christophers and Mrowietz 2003, Psoriasis. Fitzpatrick's Dermatology in General Medicine, 6th edn. McGraw-Hill, New York; pp 407-427). Induction of keratinocytes proliferation by T cells and other inflammatory cell secretions such as, cytokines, chemokines and growth factors establishes psoriasis in the skin (Cai et al. 2012, Cell. Mol. Immunol. 9:302-309).

Prevalence of Psoriatic Arthritis (PsA) varies 5%-40%. Survival analysis indicated that the incidence of PsA remained constant at 74 per 1000 person/year, whereas the prevalence increased with time since diagnosis of psoriasis, reaching 20.5% after 30 years (Gladman 2009, Rheumatol. 36:4-8. Christophers et al. 2010, J. Eur. Acad. Dermatol. Venereol. 24:548-554). A population study reported the annual incidence of PsA per 100,000 to be 7.2. The incidence increased from 3.6 between 1970 and 1979 to 9.8 between 1990 and 2000 (Wilson et al. 2009, J. Rheumatol. 36:361-367).

In a field trial to test an amastigotes polyvalent vaccine VT AS100-1 prepared with four leishmania species: L(L)amazonensis(La); L(L)venezuelensis(Lv); L(V)brasiliensis (Lb) and L(L)chagasi(Lch), for immunoprophylaxis of cutaneous leishmaniasis (CL) in Caracas, Venezuela (O'Daly et al. 1995, Gaceta Médica de Caracas 103:133-177. O'Daly J A et al. 1995, Gaceta Médica de Caracas 103:327-357. O'Daly et al. 2013, Journal Parasitology Research in press), we found that one volunteer had 100% clinical remission of plaque psoriasis, after the third vaccination. This was a natural double-blind serendipity finding since both, the volunteer and the author did not know of any effects the vaccine would have on psoriasis.

Subsequently, VT AS100-1 was evaluated in 2,770 psoriatic patients with an open label, single center study and included plaque (79.1%), guttate (10%), plaque and guttate (10.1%), palm/plantar (0.3%), erythrodermia (1.8%), inverse (0.8%), plaque and arthritis (3.4%) and nail psoriasis (0.3%). Baseline PASI compared with post-treatment values were: PASI 100: 23%; PASI 75: 45%; PASI 50: 13%; PASI 10: 9% and <PASI 10: 3% of patients respectively. For economic reasons, 7% volunteers quit treatment. Three patients in the study group had numerous skin papillomas (>10) that disappeared after remission of skin lesions in the PASI 100 group. Adverse events were mild in severity, at the site of injection. Results were confirmed in a double-blind, placebo-controlled, parallel group study, of multiple doses of VT AS100-1, which demonstrated a favorable benefit/risk profile (O'Daly et al. 2009, Arch. Dermatol. Res. 301:1-13).

Peripheral blood mononuclear cells (PBMC) lymphocyte subsets (LS) varied with PASI range (1-10, 11-20, 21-72). Pretreatment absolute values of gated LS: CD4+CD8−, CD3+CD8−, CD8+CD3+, CD8+CD4− and CD8+HLA− decreased in PBMC as PASI increased, suggesting migration from the blood to the skin. Contrary to the previous finding, the following LS: CD8+HLA+, HLA+CD8−, CD8+CD3−, CD8+CD4+, CD19, and membrane surface immunoglobulin (Ig) IgA+, IgD+, IgM+, IgE+ and IgG+ increased in PBMC as PASI increased, suggesting activation and proliferation by unknown antigens in skin. After treatment with seven doses of VT AS100-1, the following LS: CD4+CD8−, CD3+CD8−, CD8+CD3−, CD8+HLA+ and HLA+CD8−, IgD+, IgM+, IgA+, IgE+ increased, while CD8+CD3+, CD8+HLA−, CD19+, CD8+CD4+, and IgG decreased in PBMC suggesting lower sensitization and decreased lymphocyte trafficking from blood to skin as psoriasis disappeared (O'Daly et al. 2010, Arch. Dermatol. Res. 302: 95-104).

To understand the effect of leishmania vaccine on psoriasis and related diseases it should be kept in mind that leishmania promastigotes sonicates induced in vitro proliferation and IFNγ production in PBMC from individuals that never had contact with leishmania parasites. The proliferating T cell population was CD2+ in a frequency <1:10,000. Response to leishmania antigens was abolished after depletion of CD45RO+ memory cells from PBMC (Kemp et al. 1992, Infect. Immun. 60:2246-2251). After treatment with VT AS100-1 vaccines, both lymphocytes cycles between skin, and blood decreased, explaining remission of lesions. VT AS100-1 vaccine could be blocking CD2+ receptor in the immunological synapse and subsequently the inflammatory process in skin and joints (O'Daly et al. 2009, Arch. Dermatol. Res. 301:1-13. O'Daly et al. 2011, Arch. Dermatol. Res. 303:399-415).

A second single blind trial with four monovalent leishmania extracts: La, Lb, Lv and Lch, second generation vaccines VT AS100-2 in 26 subjects, resulted in remission of psoriasis. VT AS100-2 vaccines were further purified, resulting in seven Diethyl-Amino-Ethyl (DEAE) chromatography fractions VT AS200 per Leishmania specie. In vitro testing of the fractions by human PBMC blastogenesis resulted in subjects being categorized as low or high responders before treatment. Subsequently, a single blind trial in 55 subjects treated with VT AS200 fractions from Lb also had highest PASI remissions with fractions 2, 3 and 4. Two HIV+ subjects with plaque psoriasis had remission after treatment with VT AS100-1 (O'Daly et al. 2009, Arch. Dermatol. Res. 301:411-427).

Thus, there are factors in leishmania species which induce remission of psoriasis and related diseases by stimulating lymphocytes. Normal T cells and normal Ig from healthy volunteers that never experienced leishmania infection reacted with DEAE fractions from leishmania amastigotes and suppressed psoriasis by stimulating lymphocytes, contrary to the immunosuppressive effect of drug products used today (O'Daly et al. 2009, Arch. Dermatol. Res. 301:411-427). In patients with extensive lesions, a significant reduction in total T cells, T helper (TH) and T suppressor cells (TS) were observed in PBMC. The skin TH/TS ratio was greater in late guttate and in chronic plaques than the corresponding ratio in the blood, which suggests selective recruitment of activated TH cells into established psoriatic lesions (Baker et al. 1984, Br. J. Dermatol. 110:37-44), similar to data found in LS in our work (O'Daly et al. 2010, Arch. Dermatol. Res. 302:95-104. O'Daly et al. 2011, Arch. Dermatol. Res. 303:399-415).

A single blind trial on 3,132 psoriatic patients revealed 508 (16.2%) subjects with PsA that received VT AS100-1 vaccine. Baseline PASI compared with post-treatment values, were: PASI 100: 54.1%; PASI 75: 23%; PASI 50: 14.4%; and PASI 10: 8.5% of subjects, respectively. The number of VT AS100-1 doses required for 100% remission was 9.9±4.8. All groups decreased in arthritis score (AS), tender joints counts and nail changes after treatment; the highest decreased in the PASI 100 group. Relapses of psoriasis and PsA had PASI and AS lower than initial values before treatment. Clinical remissions were at lower doses and less time, after the second course of treatment. Approximately 2,599 subjects (83%) experienced at least one adverse event, injection site related and including: pain 43%, nodule formation 23%, heat 21%, and erythema 14%, similar to findings in the psoriasis AS100-1 trial mentioned above (O'Daly et al. 2011, Arch. Dermatol. Res. 303:399-415).

PsA patients PBMC also had LS varied with PASI range but in different proportions to patients with psoriasis alone, with majority being CD8+T cells. Pre-treatment, absolute values of gated LS: CD8+HLA−, CD8+HLA+, CD8+CD3−, CD8+CD3+, CD4+, decreased in PBMC as PASI increased, suggesting migration from the blood to the skin and joints. Contrary to the previous finding, the following LS: CD8+CD4−, CD3+CD8−, HLA+CD8−, CD19, CD8+CD4+and membrane surface IgA+, IgD+, IgM+, IgE+, and IgG+ increased in PBMC as PASI increased suggesting activation and proliferation by unknown antigens creating a cycle between skin/joints and peripheral blood. After nine doses of VT AS100-1, the following LS: CD8+HLA−, CD8+HLA+, CD8+CD3−, CD8+CD3+, CD8+CD4−, CD8+CD4+, CD3+CD8−, CD4+CD8−, HLA+D8−, CD19+, IgA+, IgD+, IgM+, IgE+, and IgG+ decreased significantly as compared with values before treatment. The LS decreased stopped the cycle between skin/joints and blood explaining remission of lesions (O'Daly et al. 2011, Arch. Dermatol. Res. 303:399-415). CD8+ T cells found predominantly in PsA were activated by antigens in synovium and joint cells. Thus, VT AS100-1 antigenic fractions could also stimulate regulatory CD8+ T cells in skin, synovium, and vascular wall lesions; to control the inflammatory process in the affected tissues.

Leishmania AS200 DEAE antigenic fractions 3 and 4 induced the highest delayed type hypersensitivity (DTH) reactions and % PASI reduction in psoriatic patients and also induced linear DTH reactions over a 1-40 μg dose range, in guinea pigs. When a DBA-1 mouse collagen induced arthritis (CIA) a model for human rheumatoid arthritis (RA) was used to compare AS200 fractions 3+4 treatment against: a polyvalent vaccine (VT AS100-1), a monovalent vaccine (VT AS100-2) and placebo. The VT AS200 treated mice had the least amount of forepaw inflammation and the lowest mean arthritis scores (MAS) with values even lower than dexamethasone from 7 to 20 days of follow up (O'Daly et al. 2010, Arch. Dermatol. Res. 302:567-581)

Inflammatory markers C-reactive protein (CRP) and complement 5a (C5a) assayed in PsA patients decreased significantly in serum after treatment with 6 doses of AS200 DEAE fractions 3+4 amastigotes antigens. Both patients had PASI reduction 61.6% and 66.4% respectively and biopsies with excellent improvement with decrease epidermal layer and absence of inflammatory cells in epidermis and dermis. PBMC of patients and controls were stimulated with concanavalin A (ConA) and ConA+AS100-2-Lch antigens. The Leishmania antigens decreased markedly the TNFα concentration in supernatants from patients' PBMC after treatment. In mice ConA induced hepatitis subcutaneous injection of AS100-2-Lch antigens decreased serum TNFα. Serum IL-1β in ConA induced hepatitis in mice decreased significantly after treatment with Lb VT AS200 antigens, or polyvalent VT AS100-1 vaccine, similar to dexamethasone. Interestingly, in vitro proliferation of cutaneous T cell lymphoma cells at different concentrations of AS100-2-Lch decreased as compared to PBS after 24 hours of culture in a dose-response relationship (O'Daly J A 2012, Psoriasis, a Systemic Disease Beyond the Skin, as Evidenced by Psoriatic Arthritis and Many Comorbidities—Clinical Remission with a Leishmania Amastigotes Vaccine, a Serendipity Finding pp 1-56, In Tech publishers. Croatia).

VT AS100-1 vaccine had effects on other psoriasis related skin diseases. Sixteen volunteers, with histopathological diagnosis of Atopic Dermatitis, 56.25% females, average age 24±15 years old, received 500 μg/dose AS100-1, intramuscularly every 30 days. The disease was characterized by itching, wounds, bleeding, erythema, scaling, in head, trunk, arms and legs, with variations among volunteers. The response after treatment was 90-100% regression of lesions in 62.5% of volunteers (10 out of 16), while 37.5% had around 25-50% regression of lesions as detailed in example 7. Additionally six volunteers were similarly treated with VT AS100-1. All had histopathological diagnosis of Seborrheic Dermatitis, 4 females, and 2 males, average age 33±8 years old. Three volunteers had more than 75% remission of lesions and three around 50% decrease of lesions after treatment as detailed in example 8.

Analysis of amino acid sequences, based on net charge and hydrophobicity, has been applied to small globular folded proteins and natively unfolded ones, which are specifically localized within a unique region of charge-hydrophobicity phase space and indicate that a combination of low overall hydrophobicity and large net charge represent a unique structural feature of natively unfolded proteins (Uversky et al. 2000, Proteins 41:415-427). VT AS200 peptides had, low or high hydrophobicity and large negative or positive charge similar to natively unfolded proteins. These conformations increased the interactions of peptides with synaptic complex receptors, APC, CD4+, CD8+ T cells, B cells in skin and joints inducing a prophylactic and/or immunotherapeutic effects as shown in our work.

Cationic antimicrobial peptides (CAP) occur as important innate immunity agents in many organisms, including humans, and offer a viable alternative to conventional antibiotics, as they disrupt bacterial membranes, leading to membrane lysis and cell death (Yin et al. 2012, J. Biol. Chem. 287:7738-7745). The innate immune system employs CAP as the first line of defense against the invasion of pathogens targeting, gram-positive and negative bacteria, fungi, and protozoa (Leptihn et al. 2010, Biochemistry 49:9161-9170). Similar CAP with high and low hydrophobicity has been found in TLCK treated amastigotes VT vaccines which could interact with infected cells as well as amastigotes in the intercellular space, inducing parasites' death. Gp63 primed with Hsp70 had significant protection, with higher DTH to leishmanine and reduction in parasite load of immunized animals (Yao et al. 2003, Mol. Biochem. Parasitol. 132:1-16), similar to humans immunized with VT AS100-1, AS100-2 and AS200 vaccines, inducing clinical remission of CL, with higher DTH to leishmanine (O'Daly et al. 2013, Journal Parasitology Research in press), psoriasis (O'Daly et al. 2009, Arch. Dermatol. Res. 301:1-13); PsA (O'Daly et al. 2011, Arch. Dermatol. Res. 303:399-415. O'Daly 2011, J. Clin. Rheumatol. Musculoskel. Med. 2:1-11) and CIA (O'Daly et al. 2010, Arch. Dermatol. Res. 302:567-581).

Development of psoriasis and PsA is centered in the blood vessels' behavior. Both diseases start by proliferation of blood vessels after up-regulation of angiogenic factors. Clinical remission in psoriatic lesions also starts by decreased proliferation of blood vessels, after treatment with VT AS100-1 vaccine (O'Daly 2011, J. Clin. Rheumatol. Musculoskel. Med. 2:1-11). Hsp60 has been postulated as the autoantigen initiating mechanism in atherosclerosis that might play a role in triggering an autoreactive T-cell response. The first cells invading the intima have been identified as Hsp60-reactive T cells, followed by macrophages and smooth muscle cells (Wick et al. 2004, Annu. Rev. Immunol. 22:361-403). VT AS100-1 vaccine antigens were produced after a heat shock in promastigotes that become amastigotes in a liquid culture medium (O'Daly and Rodriguez 1988, Acta Trop. 45:109-126). The MW of VT AS200 (O'Daly et al. 2009, Arch. Dermatol. Res. 301:411-427) is similar to the range of Hsp host ligands (50 to 70 kDa) for toll like receptor 2 (TLR2) and could be competing with peptides in RA synovial fibroblasts or also with Hsp60 reactive T-cell receptors in the vascular wall and in skin and joints lesions, a probable mechanism inhibiting inflammation of psoriasis, PsA, and CIA.

Ig, TCR, and MHC are multimeric complexes, involved in antigen recognition and their assembly is promoted by chaperones as Hsp. Several infections and autoimmune diseases have Hsp as prominent antigens in the humoral and cellular immune response. Further support for T cell cross recognition of Hsp regions shared by the host and pathogen comes from epitope-mapping studies with a T-cell clone derived from mice primed with mycobacterial Hsp60. Data from several arthritis models such as adjuvant arthritis and CIA favor a role for Hsp60 autoimmune T cells in disease protection (Zugel and Kaufmann, 1999, Clin. Microbiol. Rev. 12:19-39). To induce CIA, we used complete Freund's adjuvant and M. tuberculosis. It is possible that treatment with VT AS200 vaccine is acting in a beneficial way similar to mycobacterial Hsp60 epitope, cross reacting with self determinants, and also competing with ligands for TLR2 thereby, decreasing CIA as found in our work, a model for human RA (O'Daly et al. 2010, Arch. Dermatol. Res. 302:567-581).

Patients with PsA and psoriasis had higher frequency of hypertension, vascular and intestinal diseases, osteoporosis, hyperuricemia, and epilepsy. Up to 7-8 comorbidities were found in both psoriasis and PsA patients in the same subject (O'Daly 2011, J. Clin. Rheumatol. Musculoskel. Med. 2:1-11). Thus, psoriasis is a systemic disease, induced by cytokines expressed in each tissue according to genetic and environmental factors due to shared inflammatory pathways (O'Daly 2012, Psoriasis, a Systemic Disease Beyond the Skin, as Evidenced by Psoriatic Arthritis and Many Comorbidities—Clinical Remission with a Leishmania Amastigotes Vaccine, a Serendipity Finding pp 1-56, In Tech publishers. Croatia). We believe development of psoriasis and PsA is centered in the blood vessels response, and that T cells migration to skin and joints are secondary phenomena that establish the lesions in respective sites. Both diseases start by proliferation of blood vessels after up-regulation of angiogenic factors. Clinical remission in psoriasis and PsA also starts by decreased proliferation of blood vessels, after treatment with VT AS100-1 antigens (O'Daly 2011, J. Clin. Rheumatol. Musculoskel. Med. 2:1-11). Thus, we postulate blood vessels open the gate for T cells into epidermis, dermis and joints which are then sensitized by autoantigens and proliferate according to the genetic background of the subject.

ATIII, a serine protease inhibitor, has broad antiviral properties, is capable of inhibiting hepatitis C virus (Asmal et al. 2012, Virology Journal, 9:226 doi:10.1186/1743-422X-9-226) and had the highest number of sequences (14%) in VT vaccine. ATIII could be acting as anti-parasites and anti-psoriasis and related diseases, inducing clinical remission of psoriasis in HIV+ patients (O'Daly et al. 2009, Arch. Dermatol. Res. 301:411-427). ATIII, serine proteinase inhibitor, serum albumin precursor and IgM heavy chain are probably proteins from fetal bovine serum used for parasites' growth. Previously we have demonstrated that proteins from the culture medium entered parasites and are localized in membranes, cytoplasm, cytoplasmic granules and lysosomes (Bretaña and O'Daly 1976, Internat. J. Parasitol. 6:379-386) explaining their presence in amastigotes vaccines.

Hamsters immunized with TLCK-treated Lb amastigotes from culture, infected with hamsters Lb amastigotes presented increase in T and B cell responsiveness to mitogens by lymph node lymphocytes, with an increased response to ConA; absence of parasites in lymph nodes after 6 weeks post-infection and a nodule 4 times smaller than that of infected control animals; which was undetectable 70 days post-infection. Hamsters pre-immunized with TLCK treated Ld amastigotes from culture did not show suppression of the blastogenic response to mitogens of spleen and lymph node cells after infection with Ld amastigotes from hamster's spleen, and survived for more than one year, whereas infected, unimmunized animals died five months after infection. Animals preimmunized with culture parasites (Lb or Ld) treated with phenyl-methyl-sulphonyl-fluoride (PMSF) and infected with Lb or Ld amastigotes did not show any protective effect (O'Daly and Cabrera 1986, Acta Trop 43:225-436). Thus, not all protease inhibitors induced prophylactic and immunotherapeutic effects; only TLCK was effective as shown in our work in CL, psoriasis, PsA and RA.

The topical treatment for plaque psoriasis incorporates the use of emollients, keratolytic agents, coal tar, anthralin, corticosteroids of medium to strong potency, and calpotriene. All of these treatments have variable efficacy, fail to prevent frequent relapses of the disease, exhibit side effects, and pose cosmetic problems of their own (Farber and Nall 1974, Dermatologica. 148:1-18). Systemic treatment has been used in patients with physically, socially, or economically disabling psoriasis that has not responded to topical treatment. The choices to date have been phototherapy or systemic drug therapy. Generally, systemic treatment has employed phototherapy with Ultraviolet B irradiation, photo chemotherapy which combines the photosensitizing drug methoxsalen with Ultraviolet A phototherapy (PUVA), methotrexate, etretinate, systemic corticosteroids, and cyclosporine. Each of these systemic treatments has variable efficacy and undesired side effects, and some of them are very toxic and present frequent relapses of the disease. Accordingly, there is at present a need for an effective psoriasis and related diseases treatment that avoids the disadvantages associated with the currently available topical or systemic treatments (Gudjonsson et al. 2004, Clin. Exp. Immunol. 135:1-82).

### SUMMARY OF THE INVENTION

The present invention provides compositions and methods for the treatment and clinical remission of cutaneous leishmaniasis, psoriasis, psoriatic arthritis, rheumatoid arthritis, atopic dermatitis, seborrheic dermatitis and skin papilloma. In one aspect, polypeptides derived from particulate antigens of protozoans of the genus Leishmania are provided that can generate an immune response in an individual resulting in abatement of the clinical symptoms of cutaneous leishmaniasis, psoriasis, psoriatic arthritis, rheumatoid arthritis, atopic dermatitis, seborrheic dermatitis and skin papilloma. In one embodiment of this aspect, the polypeptides comprise at least one of the amino acid sequences recited in Table 1, SEQ ID NO:1 to SEQ ID NO:114 or immunogenic variants thereof. An immunogenic variant of an amino acid sequence may be either a truncated version of the sequence that retains a substantial amount of the activity of the original, or an altered version of the sequence retaining such activity and having conservative amino acid substitutions and/or modifications.

In another embodiment of this aspect, the polypeptides comprise at least an immunogenic portion of an amino acid sequence of a protein, or an immunogenic variant thereof, the protein isolated from protozoans of the genus Leishmania and having an apparent molecular weight of 20 to 140 kDa as determined by sodium dodecyl-sulfate polyacrylamide gel electrophoresis (SDS-PAGE).

In yet another embodiment of this aspect, the polypeptides comprise at least an immunogenic portion of an amino acid sequence of a protein, or an immunogenic variant thereof, the protein isolated from protozoans of the genus Leishmania and having an apparent molecular weight after total reduction and alkylation of 20 to 140 kDa as determined by SDS-PAGE.

In related aspects, the present invention provides nucleic acid sequences encoding such polypeptides, vectors incorporating such nucleic acid sequences, methods for the production of such polypeptides by transformation, transfection, or transduction of microbial host cells, and microbial host cells transformed by such vectors, transfected by such nucleic acid sequences, or transduced with such nucleic acid sequences.

In another aspect of the invention, vaccines incorporating one or more polypeptides of the present invention are provided. These vaccines may be polyvalent or monovalent, and may incorporate an adjuvant such as alumina to enhance the immune response obtained from an inoculated subject. In a preferred embodiment, the polyvalent vaccine contains polypeptide isolates from a mixture of four species of Leishmania, namely, L.(L)amazonensis, L.(L)venezuelensis, L.(V)brasiliensis, and L.(L)chagasi. Also in a preferred embodiment, the monovalent vaccine contains a polypeptide isolate from one of these four species.

In another aspect of the invention, methods for the treatment and clinical remission of cutaneous leishmaniasis, psoriasis, psoriatic arthritis, rheumatoid arthritis, atopic dermatitis, seborrheic dermatitis and skin papilloma are provided. In one embodiment, such a method involves administration of a therapeutically effective amount of a pharmaceutical composition comprising one or more polypeptides of the present invention to a subject in order to induce an immune response resulting in abatement of the clinical symptoms of cutaneous leishmaniasis, psoriasis, psoriatic arthritis, rheumatoid arthritis, atopic dermatitis, seborrheic dermatitis and skin papilloma. In a related embodiment, the method involves administration of a therapeutically effective amount of a pharmaceutical composition comprising one or more nucleic acid sequences of the present invention to a subject. The term “therapeutically effective” as used herein means a reduction of approximately 70-100% of Psoriasis Area and Severity Index (PASI) and the Arthritis Score (AS) as is understood to those skilled in the art.

Other aspects of the invention include the use of the nucleic acid sequences of the invention as probes for genetic analysis and as nucleic acid molecular weight markers, and the use of the polypeptides of the invention as molecular weight markers.

### DETAILED DESCRIPTION OF THE INVENTION

The present invention concerns novel compositions and methods for the treatment and clinical remission of cutaneous leishmaniasis, psoriasis, psoriatic arthritis, rheumatoid arthritis, atopic dermatitis, seborrheic dermatitis and skin papilloma. The compositions comprise immunogenic polypeptides or the nucleic acids encoding them. The polypeptides of the present invention can elicit an immune response in a “warm-blooded animal” including humans.

In one embodiment of the invention, the subject polypeptides can be isolated from Leishmania protozoa and, preferably, from killed Leishmania amastigote protozoa. The polypeptides of the subject invention can be obtained from protozoa of the Leishmania genus using standard protein isolation procedures which are known in the art. Also contemplated by the present invention are vaccines and pharmaceutical compositions incorporating the immunogenic polypeptides of the present invention. In one embodiment, a first-generation polyvalent vaccine for immunoprophylaxis or immunotherapeutic agent is provided, comprising a polypeptide isolate of a mixture of a plurality of Leishmania species, such as L(L)amazonensis, L(L)venezuelensis, L(V)brasiliensis, L(L)chagasi, L(L)donovani, L(L)infantum, L(L)major, L(L)panamensis, L(L)tropica, and L(L)guyanensis. Preferably, the mixture comprises L(L)amazonensis, L(L)venezuelensis, L(V)brasiliensis, and L(L)chagasi. Most preferably, the mixture consists of these four species. The organisms are preferably cultivated in the amastigote stage in the synthetic culture medium specified in Table 1, supplemented with 5% fetal bovine serum, typically at about 30-34° C., (O'Daly and Rodriguez 1988. Acta Trop. 45:109-126). Subsequently, and during the stationary phase of growth, the amastigotes are subjected to a medium containing an amount of N-p-tosyl-L-Lysine chloromethyl ketone (TLCK) or a pharmacologically acceptable salt thereof effective to kill the cells and stop proteolysis by some enzymes (O'Daly and Cabrera 1986, Acta Trop. 43:225-36). The dead cells are then isolated and treated with the non-ionic detergent Nonidet P-40 (NP40) to solubilize the surface antigens, which are discarded. The particulate antigens that comprise the immunogenic polypeptides of the present invention can be collected by centrifugation following cell disruption. These polypeptides are washed with phosphate-buffered saline (PBS) and subsequently resuspended by sonication for 5 minutes at 4° C. in PBS containing alumina.

In another embodiment, a second-generation monovalent vaccine is described, comprising a polypeptide isolate of a single Leishmania species chosen from the group consisting of L(L)amazonensis, L(L)venezuelensis, L(V)brasiliensis, L(L)chagasi, L(L)donovani, L(L)infantum, L(L)major, L(L)panamensis, L(L)tropica, and L(L)guyanensis. Preferably, the single Leishmania species is chosen from the group consisting of L(L)amazonensis, L(L)venezuelensis, L(V)brasiliensis, and L(L)chagasi. Procedures for the preparation of this vaccine are otherwise identical to those disclosed above for the first-generation polyvalent vaccine and immunotherapeutic agent.

In another embodiment, the particulate antigens that comprise the immunogenic polypeptides of the present invention can be collected by centrifugation following cell disruption for preparation of a third generation vaccine. These polypeptides are washed with phosphate-buffered saline (PBS) and subsequently resuspended by sonication for 5 minutes at 4° C. in 8 M Urea, 0.025 M Tris (Tris-hydroxy-methyl-amino-methane). The polypeptides are then subjected to chromatography on a diethylaminoethyl (DEAE)-Sephadex column with a stepwise elution from 0.05-0.3 M NaCl in a solution containing 8 M Urea, 0.025 M Tris, pH 8.3. Seven protein fractions are collected, and an inoculum comprising each protein fraction is made by resuspending the polypeptides of each fraction in PBS containing alumina.

Alternatively, the subject polypeptides can be synthesized according to known procedures and techniques, or produced recombinantly by transforming a host cell with one or more of the nucleotide sequences encoding the desired polypeptides. The polypeptides can be expressed in the host cell such that they can be isolated and purified to a desired degree of purification. The subject polypeptides can be used in accordance with the subject invention as a third-generation vaccine to treat cutaneous leishmaniasis, psoriasis, psoriatic arthritis, rheumatoid arthritis, atopic dermatitis, seborrheic dermatitis and skin papilloma.

The instant invention further concerns nucleic acid sequences that can be useful in transforming appropriate host cells to cause them to produce the polypeptides of the invention; in administration to a warm-blooded animal, either directly or as part of a pharmaceutically-acceptable composition, to generate an immune response and thereby induce clinical remission of cutaneous leishmaniasis, psoriasis, psoriatic arthritis, rheumatoid arthritis, atopic dermatitis, seborrheic dermatitis and skin papilloma in the animal; as labelled probes for genetic analysis; or as nucleic acid molecular weight markers.

One of ordinary skill in the art of molecular biology can obtain nucleic acids encoding the polypeptides of the present invention in view of the teachings provided herein. For example, the polypeptides of the first-generation vaccine of the present invention have been isolated and purified from protozoa of the Leishmania genus and comprise 20-30 bands, identified by SDS-PAGE, representing distinct polypeptides having apparent molecular weights of 10 to 140, kDa, respectively. Each of these bands represents a separate polypeptide that can be isolated and sequenced in accordance with standard amino acid sequencing procedures. The polypeptides of each second-generation vaccine were purified by subjecting to chromatography on diethylaminoethyl (DEAE)-Sephadex. Two fractions having all the activity to cure psoriasis were isolated and totally reduced and alkylated by standard procedures. These fractions were subjected to electrophoresis on polyacrylamide gels to separate the constituent polypeptides listed in Table 2, and the amino acid sequence of each polypeptide was obtained by standard protein sequencing procedures. The nucleotide sequences encoding each of these polypeptides can be derived from these amino acid sequences by application of the genetic code.

Additionally, the present invention contemplates the production of large quantities of the immunogenic polypeptides of the invention via introduction of the nucleic acids encoding them to microbial host cells. The nucleic acids can be introduced directly into the genome of the host cell or can first be incorporated into a vector which is then introduced into the host. Exemplary methods of direct incorporation include transduction by recombinant phage or cosmids, transfection where specially treated host bacterial cells can be caused to take up naked phage chromosomes and transformation by calcium precipitation. These methods are well known in the art.

Exemplary vectors include plasmids, cosmids, and phages. A genomic library for a Leishmania species can be created by routine means, and DNA of interest isolated therefrom. For example, DNA of Leishmania protozoa can be isolated and restricted with known restriction enzymes. The resulting DNA fragments can then be inserted into suitable cloning vectors for introduction to a compatible host. Depending on the contemplated host, the vector may include various regulatory and other regions, usually including an origin of replication, one or more promoter regions, and markers for the selection of transformants. In general, the vectors will provide regulatory signals for expression and amplification of the DNA of interest.

Various markers may be employed for the selection of transformants, including biocide resistance, particularly to antibiotics such as ampicillin, tetracycline, trimethoprim, chloramphenicol, and penicillin; toxins, such as colicin; and heavy metals, such as mercuric salts. Alternatively, complementation providing an essential nutrient to an auxotrophic host may be employed.

Hosts which may be employed according to techniques well known in the art for the production of the polypeptides of the present invention include unicellular microorganisms, such as prokaryotes, i.e., bacteria; and eukaryotes, such as fungi, including yeasts, algae, protozoa, molds, and the like, as well as plant cells, both in culture or in plants. Specific bacteria which are susceptible to transformation include members of the Enterobacteriaceae, such as strains of Escherichia coli; Salmonella; Bacillaceae, such as Bacillus subtilis; Pneumococcus; Streptococcus; Haemophilus influenzae, and yeasts such as Saccharomyces, among others. As used herein, the term microbial host cell encompasses all of these prokaryotic and eukaryotic organisms, including plant cells, both in culture and in plants.

Universal probes can be obtained which hybridize with certain of the fragments of a DNA library, allowing identification and selection (or “probing out”) of the genes of interest, i.e., those nucleotide sequences which encode the polypeptides described as part of the present invention. The isolation of these genes can be performed using techniques which are well known in the art of molecular biology. The isolated genes can be inserted into appropriate vectors for use in the transformation of microbial host cells. In addition, these genes can be subjected to standard nucleic acid sequencing procedures to provide specific information about the nucleotide sequence of the genes encoding the subject polypeptides.

It is now well known in the art that when synthesizing a gene for improved expression in a host cell it is desirable to design the gene such that its frequency of codon usage approaches the frequency of preferred codon usage of the host cell. For purposes of the subject invention, “frequency of preferred codon usage” refers to the preference exhibited by a specific host cell in usage of nucleotide codons to specify a given amino acid. To determine the frequency of usage of a particular codon in a gene, the number of occurrences of that codon in the gene is divided by the total number of occurrences of all codons specifying the same amino acid in the gene. Similarly, the frequency of preferred codon usage exhibited by a host cell can be calculated by averaging frequency of preferred codon usage in a large number of genes expressed by the host cell. It is preferable that this analysis be limited to genes that are highly expressed by the host cell.

Thus, in one embodiment of the subject invention, bacteria, plants, or other cells can be genetically engineered, e.g., transformed with genes from protozoa of the Leishmania spp., to attain desired expression levels of the subject polypeptides or proteins. To provide genes having enhanced expression, the DNA sequence of the gene can be modified to comprise codons preferred by highly expressed genes to attain an A+T content in nucleotide base composition which is substantially that found in the transformed host cell. It is also preferable to form an initiation sequence optimal for said host cell, and to eliminate sequences that cause destabilization, inappropriate polyadenylation, degradation and termination of RNA and to avoid sequences that constitute secondary structure hairpins and RNA splice sites. For example, in synthetic genes, the codons used to specify a given amino acid can be selected with regard to the distribution frequency of codon usage employed in highly expressed genes in the host cell to specify that amino acid. As is appreciated by those skilled in the art, the distribution frequency of codon usage utilized in the synthetic gene is a determinant of the level of expression.

Assembly of the genes of this invention can be performed using standard technology known in the art. A structural gene designed for enhanced expression in a host cell can be enzymatically assembled within a DNA vector from chemically synthesized oligonucleotide duplex segments. The gene can then be introduced into the host cell and expressed by means known in the art. Preferably, the protein produced upon expression of the synthetic gene is functionally equivalent to a native protein. According to the subject invention, “functionally equivalent” refers to identity or near identity of function. A synthetic gene product which has at least one property relating to its activity or function that is similar or identical to a natural protein is considered functionally equivalent thereto.

It is also well known in the art that the nucleotide sequences of the subject invention can be truncated such that certain of the resulting fragments of the original full-length sequence can retain the desired characteristics of the full-length sequence. A wide variety of restriction enzymes are well known by those skilled in the art to be suitable for generating fragments from larger nucleic acid molecules. For example, it is well known that Bal3l exonuclease can be conveniently used for time-controlled limited digestion of DNA. See, for example, Maniatis et al. 1982, Molecular Cloning: A Laboratory Manual, Cold Spring Harbor Laboratory, New York, pages 135-139. See also Wei et al. 1983, J. Biol. Chem. 258:13006-13512. Thus, Bal3l exonuclease (commonly referred to as “erase-a-base” procedures) allows for the removal of nucleotides from either or both ends of the subject nucleic acids, consequently generating a wide spectrum of fragments, many of which encode products that are functionally equivalent to the natural polypeptide sequences of the present invention. Labeling procedures are also well known, and the ordinarily skilled artisan could routinely screen the labeled fragments for their hybridization characteristics to determine their utility as probes. For example, it is routine to label nucleic acids for use as specific and selective probes in genetic identification or diagnostic procedures. A person of ordinary skill in the art would recognize that variations or fragments of those sequences, which specifically and selectively hybridize to the DNA of Leishmania spp., could also function as a probe. It is within the ordinary skill of persons in the art, and does not require undue experimentation, to determine whether a segment of the subject nucleic acids is a fragment or variant which specifically and selectively hybridizes in accordance with the subject invention. Therefore, fragments or variants of these nucleic acids can be useful as probes to identify, diagnose, or distinguish Leishmania species. It would also be recognized that the polynucleotides or peptides of the subject invention can be useful as molecular weight markers in respective nucleic acid or amino acid molecular weight determinations or assays.

In order to obtain a first-generation vaccine according to the subject invention, organisms of the genus Leishmania can be cultivated in synthetic culture medium comprising the ingredients listed in Table 1. In a preferred embodiment, the culture medium is supplemented with 5% fetal bovine serum. Cultivation of the protozoa according to the subject invention is typically carried out at about 30-34° C. In a particularly preferred embodiment, cultivation of the protozoa is carried out in the amastigote stage of its life cycle. The culture medium comprising the protozoan cells can then be treated in order to inactivate, and preferably kill, the cells. Upon isolation of those cells, the antigenic proteins can be purified there from and included in a pharmaceutically acceptable carrier, e.g., buffer solution, to create a second-generation vaccine. Preferably, the cells are inactivated or killed with a non-lysing agent, e.g., TLCK. The antigenic proteins of the present invention are particulate proteins that can be isolated from the cells using accepted methods. In a more specific embodiment the method of creating the second-generation vaccine of the present invention comprises the steps of (1) cultivating protozoa, preferably in the amastigote stage, in an appropriate culture medium; (2) treating said protozoan cells to inactivate or kill the cells; (3) isolating the treated cells; (4) extracting antigenic proteins from the isolated cells; and (5) formulating the second-generation vaccine composition by combining the isolated antigenic proteins in a pharmacologically acceptable carrier, e.g., phosphate buffered saline (PBS). A preferred pharmaceutically acceptable carrier is a PBS solution having alumina present within the solution.

TABLE 1
Culture medium

| Ingredient | mg/lt |
|---|---|
| CaCL₂ | 265 |
| NaHCO₃ | 2,000 |
| KCl | 400 |
| NaCl | 5,850 |
| NaH₂PO₄H₂O | 140 |
| Glucose | 1,000 |
| Hepes | 2,000 |
| Fe(NO₃)9H₂O | 0.72 |
| MgSO₄7H₂O | 200 |
| Tricine | 900 |
| D-Ribose | 10 |
| 2-Deoxy ribose | 10 |
| Adenosine-5-Triphosphate (ATP) | 5.5 |
| 2-Deoxyadenylic acid (d-AMP) | 3 |
| 5′-Thymidylic Acid (TMP) | 3 |
| 2′-Deoxicitidine-5 monophosphate (d-CMP) | 3 |
| 2′-Deoxyuridine-5-monophosphate (d-UMP) | 3 |
| 2′-Deoxyguanilic Acid (d-GMP) | 3 |
| Aspartic Acid | 120 |
| Glutamic Acid | 420 |
| Alanine | 512 |
| Arginine | 413 |
| Carnosine | 25 |
| Cysteine | 0.5 |
| Cystine | 47 |
| Glutamine | 164 |
| Glycine | 235 |
| Histidine | 6 |
| Iso-Leucine | 191 |
| Leucine | 440 |
| Lysine | 337 |
| Methionine | 140 |
| methionine-S-methyl-sulfonium chloride (U) | 0.05 |
| α-amino adipic acid | 3 |
| Asparagine | 165 |
| β-alanine | 80 |
| α-amino butyric acid | 8 |
| Phosphoethanolamine | 25 |
| Sarcosine | 57 |
| Ornithine | 3 |
| Phenylalanine | 240 |
| Proline | 248 |
| Hydroxyproline | 262.5 |
| Serine | 220 |
| Threonine | 200 |
| Tryptophan | 50 |
| Tyrosine | 210 |
| Valine | 266 |
| Ascorbic Acid | 0.05 |
| Biotin (H) | 1 |
| Carnitine | 0.05 |
| Cholecalciferol (D₃) | 0.1 |
| Choline chloride | 1 |
| Cyanocobalamine (B₁₂) | 0.01 |
| Ergocalciferol (D₂) | 0.1 |
| Folic Acid | 1 |
| Inositol | 2 |
| Menadione (K₃) | 0.01 |
| p-aminobenzoic acid | 0.05 |
| Pantothenic Acid | 1 |
| Pyridoxal | 1 |
| Pyridoxamine | 0.05 |
| Pyridoxine (B₆) | 0.025 |
| Retinol (A) | 0.14 |
| Riboflavin (B₂) | 0.1 |
| Thiamine (B₁) | 1 |
| 6,8 Thiotic acid | 0.01 |
| α-Tocoferol | 0.01 |
| 3-phytylmenadione ( K₁) | 0.01 |
| Tetrahydrofolic Acid | 0.5 |
| Hemin from Porcine | 1 |
| Taurin | 6 |
| Phosphoserine | 23 |
| Hydroxylysine | 12 |
| Citrulline | 50 |
| Niacinamide | 1 |
| Nanopure Water | 1 |
| Qsd in liters | |

### Ethic Commission

The clinical investigations were conducted in accordance with the Declaration of Helsinki. The Ethic Commission of the National Academy of Medicine of Venezuela and also the Ethic commission of the “National Institute of Scientific Investigations (IVIC)” approved the protocols for the field trials for leishmaniasis as well as, the trials for Psoriasis and related diseases. Dr. Blas Bruni Celli was appointed, trial monitor by the National Academy of Medicine of Venezuela, and oversaw all subsequent follow-up work on the trials. All volunteers signed an informed consent authorizing treatment.

To cure cutaneous leishmaniasis, psoriasis, psoriatic arthritis, rheumatoid arthritis, atopic dermatitis, seborrheic dermatitis and skin papilloma in patients with clinical and histopathological diagnosis of the disease, the first-generation polyvalent VT AS100-1 vaccine was administered intramuscularly, in the deltoid region, once a month, once every 15 days or once a week according to disease severity, for 7.6±6.0 months on average, at 500 μg/dose.

Furthermore to cure cutaneous leishmaniasis, psoriasis, psoriatic arthritis, rheumatoid arthritis, atopic dermatitis, seborrheic dermatitis and skin papilloma a monovalent VT AS100-2 immunotherapeutic agent with one of the Leishmania spp present in the first generation polyvalent VT AS100-1 vaccine was used as a subject composition with similar results to the polyvalent VT AS100-1 vaccine

Furthermore to cure cutaneous leishmaniasis, psoriasis, psoriatic arthritis, rheumatoid arthritis, atopic dermatitis, seborrheic dermatitis and skin papilloma a second-generation vaccine containing the protein fractions isolated by chromatographic means from the second-generation VT vaccine together with 0.25 ml per mg (v/w) of parasitic protein and 4 μg/ml of gentamicin was administered intramuscularly in the deltoid region once every 15 days for 3-4 doses at 200 μg/dose in 0.5 ml.

Following are examples which illustrate procedures for practicing the invention. These examples should not be construed as limiting. All percentages are by weight and all solvent mixture proportions are by volume unless otherwise noted.

### Example 1

### Preparation of the Immunogen

Organisms of the genus leishmania are cultivated in the amastigote stage in the synthetic culture medium specified in Table 1, supplemented with 5% fetal bovine serum typically at about 30-34° C. (O'Daly and Rodriguez 1988, Acta Tropica 45:109-126). For the first-generation VT AS100-1 polyvalent vaccine, amastigotes at the stationary phase of growth were collected by centrifugation at 800×g for 20 minutes at 4° C., washed in Phosphate Buffered Saline (PBS), and incubated for 3 days at 30-34° C. in Eagle's MEM (Gibco) containing 150 μg of TLCK to inactivate the parasites as described (O'Daly and Cabrera 1986, Acta Tropica 43:225-236). After two washes with PBS at 12.100×g for 10 minutes at 4° C., 1×10⁸ parasites/ml were incubated in MEM containing 0.12% Nonidet-P-40 (NP40, Sigma) for 30 minutes at 4° C. to solubilize the surface antigens which were discarded (O'Daly et al. 2009, Arch. Dermatol. Res. 301:1-13. O'Daly et al. 2011, Arch. Dermatol. Res. 303:399-415). Particulate antigens were collected by centrifugation at 12.100×g for 10 minutes at 4° C., washed twice with PBS and sonicated for 5 minutes at 4° C. in a Sonifier Cell Disrupter (Model WI 85, Heath-Systems-Ultrasonic, Inc., Plainview, N.Y.) at the microtip limit of the output control at 50 W. Protein content was determined by the method of Lowry or BCA (Lowry et al. 1951, J. Biol. Chem. 193:265-275. Smith et al. 1985, Anal. Biochem. 150:76-85). The final polyvalent first generation VT AS100-1 immunogen preparation contained 0.25 mg/ml of each Leishmania spp. antigens in PBS containing alumina (Aluminum hydroxide low viscosity gel Rehydragel, Reheis Inc., New Jersey) at the concentration 0.25 ml per mg (v/w) of parasitic protein and 4 μg/ml of gentamicin. Each step in the preparation of the immunogen was checked for sterility. The placebo dose contained 0.125 ml of Rehydragel in 0.5 ml of PBS the same amount of alumina present in the 500 μg dose of the VT AS100-1 polyvalent vaccine.

In another embodiment of the subject invention, for the second-generation VT AS100-2 monovalent vaccines, amastigotes at the stationary phase of growth were collected by centrifugation at 800×g for 20 minutes at 4° C., washed in Phosphate Buffered Saline (PBS), and incubated for 3 days at 30-34° C. in Eagle's MEM (Gibco) containing 150 μg of TLCK to inactivate the parasites as described (O'Daly and Cabrera 1986, Acta Tropica 43:225-236). After two washes with PBS at 12.100×g for 10 minutes at 4° C., 1×10⁸ parasites/ml were incubated in MEM containing 0.12% Nonidet-P-40 (NP40, Sigma) for 30 minutes at 4° C. to solubilize the surface antigens which were discarded (O'Daly et al. 2009, Arch. Dermatol. Res. 301:1-13. O'Daly et al. 2011, Arch. Dermatol. Res. 303:399-415). Particulate antigens were collected by centrifugation at 12.100×g for 10 minutes at 4° C., washed twice with PBS and sonicated for 5 minutes at 4° C. in a Sonifier Cell Disrupter (Model WI 85, Heath-Systems-Ultrasonic, Inc., Plainview, N.Y.) at the microtip limit of the output control at 50 W. Protein content was determined by the method of Lowry or BCA (Lowry et al. 1951, J. Biol. Chem., 193:265-275. Smith et al. 1985, Anal. Biochem. 150: 76-85). The final monovalent second generation VT AS100-2 immunogen preparation contained 1.0 mg/ml of each Leishmania spp. antigens in PBS containing alumina (Aluminum hydroxide low viscosity gel Rehydragel, Reheis Inc., New Jersey) at the concentration 0.25 ml per mg (v/w) of parasitic protein and 4 μg/ml of gentamicin. Each step in the preparation of the immunogen was checked for sterility. The placebo dose contained 0.125 ml of Rehydragel in 0.5 ml of PBS the same amount of alumina present in the 500 μg dose of the VT AS100-1 polyvalent vaccine.

In another embodiment of the subject invention, for the preparation of the third-generation VT AS200 vaccine particulate antigens were collected by centrifugation at 12.100×g for 10 minutes at 4° C., washed twice with PBS, dissolved in a solution containing 8 Molar Urea, 0.025 Tris (Tris-hydroxy-methyl-amino-methane) and sonicated for 5 minutes at 4° C. in a Sonifier Cell Disrupter (Model WI 85, Heath-Systems-Ultrasonic, Inc., Plainview, N.Y.) at the microtip limit of the output control at 50 W. Protein fractions were separated by DEAE-chromatography. The third-generation VT AS200 vaccine was prepared with each one of the seven protein fractions isolated after DEAE-chromatography of the subject composition containing only one leishmania specie as for example L.(V)brasiliensis or any other leishmania specie present in the first-generation VT AS100-1 polyvalent vaccine. Protein content was determined by the method of Lowry or BCA (Lowry et al. 1951, J. Biol. Chem. 193:265-275. Smith et al. 1985, Anal. Biochem. 150:76-85). Each protein fraction was dissolved in PBS and sonicated for 5 minutes at 4° C. in a Sonifier Cell Disrupter (Model WI 85, Heath-Systems-Ultrasonic, Inc., Plainview, N.Y.) at the microtip limit of the output control at 50 W. Subsequently each fraction was filter-sterilized through 0.20 μm Millipore® filters. The final immunogen preparation contained 400 μg/ml of each of the antigenic fractions in PBS containing alumina (Aluminum hydroxide low viscosity gel Rehydragel, Reheis Inc., New Jersey) at the concentration 0.25 ml per mg (v/w) of parasitic protein and 4 μg/ml of gentamicin. Each step in the preparation of the third generation immunogen was also checked for sterility. Antigens in PBS supplemented with Rehydragel The placebo dose contained 0.125 ml of Rehydragel in 0.5 ml of PBS the same amount of alumina present in the 500 μg dose of the polyvalent vaccine.

Aliquots were incubated in ESM containing 5% Fetal Bovine Serum (FBS, Gibco) and in agar plates containing 12.5% (w/v) Bacto-Peptone (Difco), 12.5% (w/v) yeast extract (Becton Dickinson), 3.75% (w/v), glucose, and 3.75% (w/v) BBL agar (Becton Dickinson). Samples were incubated for 72 hours at 37° C. to detect fast growing bacteria and for 3 weeks at 26° C. for slow growing bacteria and fungus. Each batch of the immunogen was controlled by SDS-polyacrylamide gel electrophoresis to ensure consistency in the pattern of Leishmania protein bands. Each batch from the first, second and third generation vaccines was also tested with E-Toxate (Sigma) for the presence of pyrogens. The first-generation immunogen was stable at 4° C. for at least 4 weeks.

### Example 2

### Protein Components of the Immunogen

From the immunogen preparations obtained from the procedures described in Example 1 above, 23-30 protein bands were identified via SDS-polyacrylamide gel electrophoresis of the TLCK-treated, NP-40-extracted amastigotes from L(L)amazonensis, L(L)venezuelensis, L(V)brasiliensis, and L(L)chagasi, with apparent molecular weights from 10 to 140 kDa. In untreated entire amastigotes extracts more than 50 bands with molecular weights ranging from 10 to 150 kDa were observed in each Leishmania species (O'Daly et al. 2009, Arch. Dermatol. Res. 301:411-427).

The immunogen preparations of the third-generation vaccine, which contain protein fractions 3 and 4 obtained after DEAE-chromatography and total reduction and alkylation, had six denser bands with molecular weights from 50 to 70 kDa (O'Daly et al. 2010, Arch. Dermatol. Res. 302:567-581).

### Example 3

### Safety and Immunogenicity

The immunogenic composition comprising the proteins of the first-generation VT AS100-1 vaccine, described in Examples 1 and 2, above, was injected into a human volunteer (JAO) at monthly intervals, beginning with 50 μg and increasing the dose by 50 μg each month, in order to determine the dose capable of inducing an IDR greater than 5 mm. This dose was found to be 200 μg. At both one month and six months after the last dose of vaccine, the following blood tests were performed on this volunteer. complete blood count; differential white blood cell count; urea; creatinin; sugar; alkaline phosphatase; bilirubin; transaminases; cholesterol; triglycerides; C reactive protein; serological tests such as VDRL, HIV, antinuclear antibodies, LE cells; and urine and fecal analysis. All the values were within normal limits, and no side effects were observed.

Additionally laboratory analyses as listed in Table 2 were performed in 55 psoriatic volunteers with 21.4±13.1 doses of first generation VT AS100-1 vaccine, after treatment.

TABLE 2
Laboratory analysis in 55 psoriasis patients

| | |
|---|---|
| White blood cell count/ul | 6003 ± 4165 |
| % Neutrophiles | 53.1 ± 13.3 |
| % Lymphocytes | 29.3 ± 13.3 |
| % Monocytes | 5.8 ± 3.8 |
| % Eosynophiles | 2.9 ± 2.3 |
| % Basophiles | 0.7 ± 0.6 |
| Red blood cell count × 10⁶/ul | 4.7 ± 0.6 |
| Hemoglobin g/dl | 13.3 ± 1.9 |
| Hematocrit (%) | 42.0 ± 5.9 |
| VCM(fl) | 91.6 ± 7.7 |
| MCH(pg) | 29.2 ± 3.2 |
| MCHC(g/dl) | 31.9 ± 1.0 |
| RDW-SD(fl) | 20.1 ± 14.9 |
| Platelets × 10⁶/ul | 250.3 ± 84.2 |
| Urea(mg/dl) | 19.7 ± 8.5 |
| Creatinine (mg/dl) | 0.9 ± 0.2 |
| Uric Acid (mg/dl) | 5.6 ± 1.6 |
| Blood Sugar (mg/dl) | 89.8 ± 15.1 |
| Total Protein (g/dl) | 7.2 ± 0.8 |
| Albumin (g/dl) | 3.8 ± 0.9 |
| Globulins (g/dl) | 3.3 ± 0.8 |
| Triglycerides (mg/dl) | 161.0 ± 107.1 |
| Low density lipoproteins (mg/dl) | 102.8 ± 44.5 |
| Very Low density lipoproteins (mg/dl) | 35.0 ± 23.3 |
| Lactic acid dehydrogenase (mg/dl) | 36.1 ± 13.2 |
| Protrombin time | 11.7 ± 1.3 |
| Tromboplastin partial time | 29.5 ± 6.5 |
| Oxaloacetic transaminase (u/l) | 29.0 ± 14.1 |
| Pyruvic transaminase (u/l) | 26.1 ± 15.1 |
| Sodium (mg/dl) | 144.9 ± 2.1 |
| Potassium (mg/dl) | 4.2 ± 0.3 |
| Chlorine (meq/l) | 105.3 ± 2.6 |
| Calcium (mg/dl) | 8.7 ± 0.3 |
| Phosphorus (mg/dl) | 2.9 ± 0.4 |

All the values were within normal limits, and no side effects were observed.

### Example 4

### Preparation of Immunotherapeutic Agent Compositions

For the second-generation monovalent immunotherapeutic agent, cultivated amastigotes of each species of Leishmania were collected by centrifugation at 800×g for 20 minutes at 4° C., washed in Phosphate Buffered Saline (PBS) and incubated for 3 days at 30-34° C. in Eagles's MEM (Gibco) containing 150 μg of TLCK to inactivate the parasites as described, at 1×10⁸ parasites/ml. This step is preferably carried out when the amastigotes are in the stationary growth phase, after two washes with PBS at 12.100×g for 10 minutes at 4° C.

In a particularly preferred embodiment, preparation of a protective monovalent second generation immunogenic composition according to the subject invention comprises the following steps:
A) cultivating organisms of the genus Leishmania in the amastigote state in a synthetic culture medium containing the ingredients listed in Table 1 supplemented with 5% fetal bovine serum typically at about 30-34° C.;
B) subjecting organisms of the genus Leishmania in the amastigote stage, and at the stationary phase of growth, to a medium containing an amount of N-p-tosyl-L-Lysine chloromethyl ketone (TLCK) or a pharmacologically acceptable salt thereof effective to kill said cells;
C) isolating said killed cells;
D) extracting the surface proteins with the non-ionic detergent Nonidet p-40;
E) centrifugation of the preparation to isolate particulate antigens;
F) washing twice with PBS; and
G) forming an immunizing inoculum comprising said particulate antigens from said killed cells by resuspending them in phosphate buffered saline comprising alumina.

For the third generation vaccine composition, cultivated amastigotes were collected by centrifugation at 800×g for 20 minutes at 4° C., washed in Phosphate Buffered Saline (PBS) and incubated for 3 days at 30-34° C. in Eagles's MEM (Gibco) containing 150 μg of TLCK to inactivate the parasites as described, at 1×10⁸ parasites/ml. This step is preferably carried out when the amastigotes are in the stationary growth phase, after two washes with PBS at 12.100×g for 10 minutes at 4° C.

In a particularly preferred embodiment, preparation of a protective and/or immunotherapeutic third generation AS200 immunogenic composition according to the subject invention comprises the following steps:
A) cultivating organisms of the genus Leishmania in the amastigote state in a synthetic culture medium containing the ingredients listed in Table 1 supplemented with 5% fetal bovine serum typically at about 30-34° C.;
B) subjecting organisms of the genus Leishmania in the amastigote stage and at the stationary phase of growth, to a medium containing an amount of N-p-tosyl-L-Lysine chloromethyl ketone (TLCK) or a pharmacologically acceptable salt thereof effective to kill said cells;
C) isolating said killed cells;
D) extracting the surface proteins with the non-ionic detergent Nonidet P-40;
E) centrifugation of the preparation to isolate particulate antigens;
F) washing twice with PBS,
G) dissolving in a solution containing 8 Molar Urea, 0.025 Molar Tris (Tris-hydroxy-methyl-amino-methane) and sonicating for 5 minutes at 4° C. in a Sonifier Cell Disrupter (Model WI 85, Heath-Systems-Ultrasonic, Inc., Plainview, N.Y.) at the microtip limit of the output control at 50 W.
H) separating protein fractions in a DEAE-Sephadex column with a NaCl stepwise elution from 0.05-0.3 Molar NaCl concentration in a solution containing 8 Molar Urea, 0.025 Molar Tris pH 8.3; and
I) forming an immunizing inoculum comprising said particulate antigens from said killed cells by resuspending them in phosphate buffered saline comprising alumina.

In a particularly preferred embodiment, preparation of an immunogenic composition for clinical remission of cutaneous leishmaniasis, psoriasis, psoriatic arthritis, rheumatoid arthritis, atopic dermatitis, seborrheic dermatitis and skin papilloma according to the third-generation subject invention comprises the following steps:
A) cultivating organisms of the genus Leishmania in the amastigote state in a synthetic culture medium containing the ingredients listed in Table 1 supplemented with 5% fetal bovine serum typically at about 30-34° C.;
B) subjecting organisms of the genus Leishmania in the amastigote stage and at the stationary phase of growth, to a medium containing an amount of N-p-tosyl-L-Lysine chloromethyl ketone (TLCK) or a pharmacologically acceptable salt thereof effective to kill said cells;
C) isolating said killed cells;
D) extracting the surface proteins with the non-ionic detergent Nonidet p-40;
E) DEAE Sephadex chromatography of particulate antigens from only one Leishmania specie, as for example L(V)brasiliensis or any other Leishmania specie present in the first-generation vaccine;
F) isolating seven protein fractions in 8 Molar urea, 0.025 Molar Tris pH 8.3, separated using stepwise elution with 0.05-0.3 Molar NaCl;
G) dialysis vs. distilled water and lyophylization of protein fractions;
H) dissolving the protein fractions in phosphate buffered saline;
I) determining protein content of the fractions by the method of Lowry or BCA (Lowry et al. 1951, J. Biol. Chem. 193:265-275. Smith et al. 1985, Anal. Biochem. 150:76-85).
J) sonicating each protein fraction in phosphate buffered saline for 5 minutes at 4° C. in a Sonifier Cell Disrupter (Model WI 85, Heath-Systems-Ultrasonic, Inc., Plainview, N.Y.) at the microtip limit of the output control at 50 W;
K) passing each fraction through 0.20 μm Millipore® filters; and
L) forming a third-generation immunizing inoculum comprising one or more of said protein fractions by resuspending the one or more fractions in phosphate buffered saline containing alumina.

### Example 5

### Formulation and Administration

The compounds of the invention are useful for various purposes, both therapeutic and non-therapeutic. Therapeutic application of the new compounds and compositions containing them can be contemplated to be accomplished by any suitable therapeutic method and technique presently or prospectively known to those skilled in the art. Further, the compounds of the invention have utility as starting materials or intermediates for the preparation of other useful compounds and compositions.

The dosage administered to a host in the above indications will be dependent upon the identity of the infection, the type of host involved, including the host's age, weight, and health, the existence and nature of concurrent treatments, if any, the frequency of treatment, and the therapeutic ratio.

The compounds of the subject invention can be formulated according to known methods for the preparation of pharmaceutical compositions. Formulations are described in detail in a number of sources which are well known and readily available to those skilled in the art. For example, Remington's Pharmaceutical Science by E. W. Martin describes formulations that can be used in connection with the subject invention, and is hereby incorporated by reference. In general, the compositions of the subject invention will be formulated such that an effective amount of the bioactive compound(s) is (are) combined with a suitable carrier in order to facilitate effective administration of the composition.

### Example 6

### Identification and Characterization of Proteins and Peptides that Induce Clinical Remission of Cutaneous Leishmaniasis, Psoriasis, Psoriatic Arthritis, Rheumatoid Arthritis, Atopic Dermatitis, Seborrheic Dermatitis and Skin Papilloma

Amino acid sequences were done at the Harvard Microchemistry Facility; Harvard University 16 Divinity Ave. Cambridge Mass. 02138. AS200 fraction three+four, from Lb at 50:50 concentration each, were electrophoresed in invitrogen 4-20% NuPAGE Tris-Glycine SDS gels under reducing conditions with Tris-glycine SDS running buffer in an XCell SureLock Mini-Cell (Invitrogen life technologies) following the manufacturer's instructions, and the gels send to be sequenced at Harvard's facility. The sequences summarized here are the result of Trypsin_Strict proteolytic digestion followed by a single micro capillary reverse-phase HPLC nano-electrospray tandem mass spectrometry (μLC/MS/MS) on a Finnigan LCQ DECA XP Plus quadrupole ion trap mass spectrometer. This instrument configuration is capable of acquiring individual sequence (MS/MS) spectra on-line at high sensitivity (<<<1 femtomole) for multiple peptides in the chromatographic run. These MS/MS spectra, (also referred to as CID, sequence or fragmentation spectra), are then correlated with known sequences using the algorithm Sequest developed at the Univ. of Washington (Eng J K et al. 1994 J. Am. Soc. Mass Spectrom. 5, 976-989) and programs developed in our laboratory (Chittum et al. 1998, Biochemistry 37, 10866-10870). MS/MS peptide sequences are then reviewed by a scientist for consensus with known proteins and the results manually confirmed for fidelity.

TABLE 3
Amino acid sequences of peptides

| Protein in AS200 | | Pep | Peptide | Pep |
|---|---|---|---|---|
| (Percent/114 peptides) | 100% Homology sequence in AS200 peptides | # | charge | Hydrop |
| Antithrombin-III | GluGlnLeuGlnAspMetGlyLeuGluAspLeuPheSerProGluLys | 1 | −−−−−+ | −15.1 |
| (ATIII), Serine | ThrSerAspGlnIleuHisPhePhePheAlaLys | 2 | −+ | -0.9 |
| protease inhibitor | PheArgIleuGluSerPheSerValLys | 3 | +−+ | 0.8 |
| (serpin), Bovine | AsnAsnAsnAspAsnIleuPheLeuSerProLeuSerIleuSerThr | 4 | −+ | 0.9 |
| (14) | AlaPheAlaMetThrLys | | | |
| | SerArgLeuProGlyIleuValAlaGluGlyArg | 5 | +−+ | −1.4 |
| | SerSerGluLeuValSerAlaAsnArg | 6 | −+ | -3.9 |
| | AlaPheLeuGluValAsnGluGluGlySerGluAlaAlaAlaSerThr | 7 | −−−−+ | 7.1 |
| | ValIleuSerIleuAlaGlyArg | | | |
| | LeuGlnProLeuAspPheLys | 8 | −+ | -2.1 |
| | LeuProGlyIleuValAlaGluGlyArg | 9 | −+ | 4.3 |
| | LysAlaThrGluGlyGlnGlySerGluGlnLysIleuProGlyAla | 10 | +−−++ | -26.7 |
| | ThrAsnArg | | | |
| | ArgValTrpGluLeuSerLys | 11 | +−+ | -5.2 |
| | IleuThrAspValIleuProProGlnAlaIleuAsnGluPheThr | 12 | −−+ | 23.1 |
| | ValLeuValIleuValAsnThrIlepheLys | | | |
| | SerLysPheSerProGluAsnThrArg | 13 | +−+ | -16.5 |
| | ValGluGlnGluLeuThrProAspMetLeuGlnGluTrpLeuAspGlu | 14 | −−−−−−−+ | -6.2 |
| | LeuThrGluThrLeuLeuValValHisMetProArg | | | |
| | PheAspThrIleuSerGluLys | 15 | −−+ | -5.1 |
| | AlaThrGluGlyGlnGlySerGluGlnLysIleProGlyAlaThrAsn | 16 | −−++ | -22.8 |
| | Arg | | | |
| Major surface glyco- | HisLeuValProGlnAlaLeuGlnLeuHisArg | 17 | + | -2.1 |
| protein GP63, | ValLysLeuSerSerValSerAspAlaPheGlnLys | 18 | +−+ | -0.1 |
| Leishmanolysin C1 | CysIleuAspGlyAlaPheThrProLys | 19 | −+ | 1.5 |
| precursor | TyrAspGlnLeuValThrArg | 20 | −+ | -5.5 |
| (EC 3.4.24.36) protein | LeuSerSerValSerAspAlaPheGluLys | 21 | −−+ | -0.7 |
| metalloprotease | GlyGlyTyrValThrCysProProTyrValGluValCysGlnGlyAsn | 22 | −+ | -2.8 |
| GP63, MSP cell surface | ValLys | | | |
| protease, promastigote | AsnValGlyCysAlaPheLeuSerGluLys | 23 | −+ | 3.0 |
| surface endopeptidase, | ThrTyrSerValGlnValArg | 24 | −+ | -2.4 |
| L(L) mwdcana (8.8) | AlaGluGluMetProTrpGlyArg | 25 | −−+ | -10.7 |
| | AlaAlaAsnTrpGlyAlaLeuArg | 26 | + | -0.1 |
| Heat shock protein 83 | AlaAspLeuValAsnAsnLeuGlyThrIleuAlaArg | 27 | −+ | 3.1 |
| L(L)amazonensis | GluValLeuGlyAspLysValGluLys | 28 | −−+−+ | -6.5 |
| (8.8) | GluGlyValHisPheGluGluSerGluGluGluLysArg | 29 | −−−−−−++ | -26.8 |
| | ThrThrGluLysGluValThrAspGluAspGluGluGluAlaLys | 30 | −+−−−−−−−+ | -31.9 |
| | LysAlaAspGluAspGlyGluGluProLysValGluGluValThrGlu | 31 | +−−−−−+−−−−−+ | 40.0 |
| | GlyGluGluGlyLys | | | |
| | GlyValValAspSerGluAspLeuProLeuAsnIleuSerArg | 32 | −−−+ | -1.6 |
| | ValGluGluValThrGluGlyGluGluGlyLysLys | 33 | −−−−−++ | -18.4 |
| | ThrLeuThrValGluAspAsnGlyIleuGlyMetThrLys | 34 | −−+ | -2.9 |
| | HisSerGluPheIleuGlyTyrAspIleuGluLeuMetValGluLys | 35 | −−−−+ | -1.9 |
| | ThrMetLysGluValLeuGlyAspLysValGluLys | 36 | +−−+−+ | -9.2 |
| serine (or cysteine) | SerValLeuGlyAspValGlyIleuThrGluValPheSerAspArg | 37 | −−−+ | 5.6 |
| proteinase inhibitor, | TyrAlaSerSerAlaAsnLeuHisLeuProLys | 38 | + | -3.9 |
| Bade A α-1 | GlyAsnThrHisThrGluIleuLeuLys | 39 | −+ | -7.6 |
| antiproteinase, | LeuValAspThrPheLeuGluAspValLys | 40 | −−−+ | 3.7 |
| antitrypsin, Bos | ValLeuAspProAsnThrValPheAlaLeuValAsnTyrIleuSer | 41 | −+ | 13.3 |
| Taurus (9.6) | PheLys | | | |
| | LysIleuAsnAspTyrValGluLys | 42 | +−−+ | -10.9 |
| | AspPheHisValAsaGluGlnThrThrValLys | 43 | −−−+ | -11.3 |
| | LeuGlnGlnLeuGluAspLysLeuAsnAsnGluLeuLeuAlaLys | 44 | −−+−+ | -11.5 |
| | LeuSerIleuSerGluThrTyrAspLeuLys | 45 | −−+ | -2.4 |
| | AlaAspLeuSerGlyIleuThrLys | 46 | −+ | 0.8 |
| | IleuAsnAspTyrValGluLys | 47 | −−+ | -7.0 |
| α-tubulin L(L) | GluIleuValAspLeuAlaLeuAspArg | 48 | −−−+ | 3.9 |
| donovani (4.4) | LeuSerValAspTyrGlyLysLys | 49 | −++ | -5.8 |
| | SerLeuAspIleuGluArgProSerTyrThrAsnValAsnArg | 50 | −−++ | -15.7 |
| | GlnLeuPheAsnProGluGlnLeuValSerGlyLysGluAspAlaAla | 51 | −+−−+ | -20.5 |
| | AsnAsnTyrAlaArg | | | |
| | GlyHisTyrThrIleuGlyLys | 52 | + | -5.4 |
| TUBULIN β CHAIN | IleuAsnValTyrPheAspGluSerAlaGlyGlyArg | 53 | −−+ | -4.6 |
| L(L)amazonensis | LeuAlaValAsnLeuValProPheProArg | 54 | + | 9.4 |
| (3.5) | ValGlyGluGlnPheThrGlyMetPheArg | 55 | + | -1.3 |
| | AlaValLeuMetAspLeuGluProGlyThrMetAspSerValArg | 56 | −−+ | 3.1 |
| elongation factor 1 α | SerThrAlaThrGlyHisLeuIleuTyrLys | 57 | + | -0.9 |
| L(L) donovani | ValGlyTyrAsnProGluLys | 58 | −+ | -10.0 |
| (5.3) | GluAlaAlaGluIleuGlyLys | 59 | −−+ | -3.2 |
| | SerValPheThrIleuIleuAspAlaProGlyHisArgAspPheIleuLys | 60 | −+−+ | 3.0 |
| | TyrAspGluIleuSerLys | 61 | −−+ | 4.5 |
| | AsnMetIleuThrGlyThrSerGlnAlaAspAlaAlaIleuLeuMetIleu | 62 | −−−+ | 4.9 |
| | AspSerThrHisGlyGly Phe- | | | |
| | GluAlaGlyIleuSerLys | | | |
| putative mitochondrial | AspTyrGlyValLeuIleuGluGluSerGlyIleuAlaLeuArg | 63 | −−−+ | 4.7 |
| peroxiredoxin | AsnValAspGluAlaLeuArg | 64 | −+ | -5.1 |
| L(L) infantum | GluAlaAlaProGlnPheSerGlyGlnAlaValValAsnGly | 65 | −+ | -5.9 |
| (5.3) | AlaIleuLys | | | |
| | LysLysGlyGlyLeuGlyGluMetHisIleuProValLeuAlaAspLys | 66 | ++−−+ | -4.7 |
| | GlyGlyLeuGlyGluMetHisIleuProValLeuAlaAspLys | 67 | −−+ | 3.1 |
| | GlyLeuPheIleuIleuAspLysLys | 68 | −++ | 3.9 |
| Glutamate | TyrThrGlySerValIleuAlaLeuTyrProGluPheValLys | 69 | −+ | 7.8 |
| dehydrogenase | SerSerLeuThrGlnGluValMetSerGluArg | 70 | −−+ | -8.2 |
| precursor Sauro | PheGlnThrGlyGlyProAspGlyAspLeuGlySerAsnGluValLeuArg | 71 | −−−+ | -12.1 |
| L(L)tarentolae (3.5) | LeuProAspGlyThrLeuValGluAspGlySerArg | 72 | −−−+ | -7.1 |
| Cysteine proteinase B | GluSerAlaAlaProGlyThrSerThrSerSerGluThrProAlaProArg | 73 | −−+ | -16.6 |
| precursor L(L)mexicana | AsnGlyProIleuAlaIleuAlaLeuAspAlaSerSerPheMetSerTyr | 74 | −+ | 6.3 |
| (3.5) | Lys | | | |
| | AlaTyrGluThrLeuAlaGluGluGlnGlnArg | 75 | −−−+ | -16.6 |
| | AsnProHisAlaGlnPheGlyIleuThrLys | 76 | + | -7.7 |
| IgM heavy chain | IleuValSerGlyIleuSerGluGlyGlnValGluThrValGlnSerSer | 77 | −−+ | -18.0 |
| constant region | ProValThrPheArg | | | |
| Bos Taurus | AlaLeuGluThrHisThrTyrPheGluArg | 78 | −−+ | -9.0 |
| (3.5) | PheTrpThrPheProGluValLeuArg | 79 | −+ | 2.4 |
| | AspValAlaMetLysProProSerValTyrLeuLeuProProThrArg | 80 | −++ | -1.4 |
| Serum albumin | HisLeuValAspGluProGlnAsnLeuIleuLys | 81 | −−+ | -6.4 |
| precursor Bos taurus | LysValProGlnValSerThrProThrLeuValGluValSerArg | 82 | +−+ | -1.0 |
| (3.5) | LeuValAsnGluLeuThrGluPheAlaLys | 83 | −−+ | 1.3 |
| | AlaGluPheValGluValThrLys | 84 | −−+ | 1.4 |
| Alpha 2HS glycoprotein | HisThrLeuAsnGlnIleuAspSerValLys | 85 | −+ | -6.6 |
| Bos Taurus | GlnAspGlyGlnPheSerValLeuPheThrLys | 86 | −+ | -2.7 |
| Fetuin A | AlaLeuGlyGlyGluAspValArg | 87 | −−+ | -2.5 |
| (3.5) | GluValValAspProThrLys | 88 | −−+ | 4.8 |
| peroxidoxin 1 | LeuLeuGluAlaPheGlnPheValGluLys | 89 | −−+ | 4.8 |
| L(L)chagasi | GlnIleuThrValAsnAspMetProValGlyArg | 90 | −+ | -2.9 |
| (2.6) | AsnValGluGluValLeuArg | 91 | −−+ | -2.8 |
| Heat shock protein70, | ValValAsnGluProThrAlaAlaAlaLeuAlaTyrGlyMetAspLys | 92 | −−+ | 3.3 |
| L(L) major (1.8) | GluIleuAsnAspValValLeuValGlyGlyMetThrArg | 93 | −−+ | 6.3 |
| Disulfide isomerase PDI | SerGlnValLeuLeuThrTyrIleuAspGlyAspGlnTyrArgProVal | 94 | −−++ | -9.4 |
| | SerArg | | | |
| L(L)major (1.8) | ThrAlaAlaGlyIleuAlaSerTyrMetLys | 95 | + | 4.7 |
| Polyubiquitin5 | IleuGlnAspLysGluGlyIleuProProAspGlnGlnArg | 96 | −+−−+ | -24.0 |
| Dictyostelium | ThrLeuSerAspTyrAsnIleuGln | 97 | − | -5.0 |
| discoideum (1.8) | | | | |
| ADP-ribosylation factor | LeuGluGluThrAlaAlaGluLeuAspAlaLeuLeuGlnGluGluArg | 98 | −−−−−−+ | -9.1 |
| L(L) amazonensis | ArgLeuGluGluThrAlaAlaGluLeuAspAlaLeuLeuGlnGluGlu | 99 | +−−−−−−+ | -13.6 |
| (1.8) | Arg | | | |
| Myo-Inositol-1- | AlaProAlaValProGluGlyThrProValValAsnAlaLeuAsnArg | 100 | −+ | 0.9 |
| phosphate synthase | SerAsnSerValLeuPheProGluGlyAlaArg | 101 | −+ | -2.5 |
| L(L)mexlcana (1.8) | | | | |
| Ecto-metalloproteinase | IleuAlaValSerAlaGluAspLeuThrAspProAlaTyrHisCysAla | 102 | −−−+ | -0.4 |
| L(L)amazonensis (1.8) | Arg | | | |
| | AlaGluAlaMetProTrpGlyArg | 103 | −+ | -6.4 |
| Hexokinase L(L)major | SerAlaLeuValGlyAspAlaThrAspLeuPheAspPheIleuAlaGln | 104 | −−−+ | -18.2 |
| (0.9) | SerValArg | | | |
| ATPase β subunit | PheThrGlnAlaAsnSerGluValSerAlaLeuLeuGlyArg | 105 | −+ | 0.6 |
| Trypanosoma brucei | | | | |
| (0.9) | | | | |
| Single strand-specific | TyrGlnArgProLeuSerTyrThrAspLeuPheAlaLeuSerAlaThr | 106 | +−−+ | −9.9 |
| nuclease L(L)pifanoi | AlaAspArg | | | |
| (0.9) | | | | |
| Endo cytokeratin Homo | SerLeuAsnAsnLysPheAlaSerPheIleuAspLysValArg | 107 | +−++ | -4.5 |
| Sapiens (0.9) | | | | |
| NADP-specific iso- | ThrLeuGluAlaGluAlaAlaHisGlyThrValThrArg | 108 | −−+ | -3.8 |
| citrate dehydrogenase | | | | |
| L albus (0.9) | | | | |
| Hypothetical protein | AlaGluLeuGluAlaGlnValAlaArg | 109 | −−+ | -1.6 |
| Chr3_0210 L(L)major | | | | |
| (0.9) | | | | |
| GM05016p Drosophila | SerGlyGlnValValProGlyTyrGlyHisAlaValLeuArg | 110 | + | 2.7 |
| melanogaster (0.9) | | | | |
| Cathepsin B Cys | HisValSerGlyAspHisLeuGlyGlyHisAlaValLys | 111 | −+ | 4.0 |
| protease L(L)mexicana | | | | |
| (0.9) | | | | |
| Eukaryotic initiation | ValLeuValLeuAspGluAleAspGluMetLeuSerGlnGlyPheAla | 112 | −−−−−−+ | 4.9 |
| factor 4A Ribosomal | AspGlnILeuTyrGluIleuPheArg | | | |
| protein (0.9) | | | | |
| Alpha-2-plasmin | GlyGluAspLeuAlaAsnIleuAsnArg | 113 | −−+ | 4.8 |
| inhibitor Bos Taurus | | | | |
| (0.9) | | | | |
| CLUS_BOVIN Clusterin | LeuTyrAspGlnLeuLeuGlnSerTyrGlnGlnLys | 114 | −+ | -13.4 |
| precursor Bos Taurus | | | | |
| (0.9) | | | | |

Uncharged peptides are in black, cationic peptides in red, anionic peptides in green, Pep: Peptide. Hydrop: Hydropathy

### Example 7

A field trial for Atopic Dermatitis in 16 volunteers, 56.25% females average age 24±15 years old range 4-47 years old, with 4.9±4.1 doses VT AS100-1 polyvalent vaccine also had a positive benefit/risk ratio in 6 years of follow up (Table 4).

TABLE 4
ATOPIC DERMATITIS

| Case | | | Treatment Start | Treatment End | # of Doses | | Description of Clinical Effect After |
|---|---|---|---|---|---|---|---|
| # | Age | Sex | Date | Date | Total | Baseline Diagnosis | Treatment |
| 157 | 34 | M | 29-Jun-94 | 9-Jul-96 | 18 | Severe general Atopic | No scaling, no itching, 90% |
| | | | | | | dermatitis started 3 years | regression of lesions after 4 doses, |
| | | | | | | ago, itching, abundant | relapse after 5 doses, 95% |
| | | | | | | scaling | regression after 12 doses, 100% after |
| | | | | | | | 14 doses. Slight relapse with final |
| | | | | | | | 90% regression after 18 doses |
| 264 | 10 | M | 24-Feb-95 | 31-Mar-95 | 3 | Face, arms, legs | After 1 dose, only small lesion on |
| | | | | | | | posterior leg remained, relapse after 2 |
| | | | | | | | doses with 20% of initial lesions |
| | | | | | | | reappearing, did not return after 3rd |
| | | | | | | | dose |
| 331 | 34 | F | 15-May-95 | 15-Jun-95 | 2 | Itching, pain, scaling of | Itching, no pain, slight scaling, no |
| | | | | | | wounds, bleeding on hands | wounds, no bleeding after 1 dose |
| 476 | 47 | F | 21-Nov-95 | 13-Dec-95 | 2 | Small lesions in scalp and | Small lesions of face and scalp |
| | | | | | | face less than 2 cm. | increased first week after 1 dose and |
| | | | | | | | then decreased |
| 525 | 40 | M | 18-Jan-96 | 10-Jun-98 | 8 | Itching, scaling, redness, | After 1 dose no itching, 30% |
| | | | | | | started 2 years ago on trunk, | remission of lesions, after 2 doses |
| | | | | | | back, abdomen, legs | relapse similar to baseline, after 3 |
| | | | | | | | doses arms are better, legs minor |
| | | | | | | | lesions, after 4 doses 100% remission |
| | | | | | | | of lesions continuing thru 7 doses |
| 605 | 10 | F | 19-Mar-96 | 8-Oct-96 | 7 | Itching wounds, scaling, | 20% regression of lesions after 2 |
| | | | | | | redness, under arms (axils) | doses, no redness itching or wound |
| | | | | | | eyebrows, eyes | after 3 doses, 90% of regression after |
| | | | | | | | 5 doses, minor relapse after 6 doses |
| 722 | 45 | M | 23-Jul-96 | 7-Oct-96 | 3 | Red plaques with scaling in | 75% decrease in all lesions after 1 |
| | | | | | | scalp with itching, lesions in | dose. No itching, no erythema, no |
| | | | | | | legs | plaques. All lesions decreased 90% |
| | | | | | | | after 2 doses |
| 807 | 42 | F | 3-Oct-96 | 21-Nov-96 | 2 | Multiple lesions in legs, | 90% regression of lesions in legs after |
| | | | | | | papule, and inflammatory | 1 dose |
| | | | | | | nodules, less than 1 cm. in | |
| | | | | | | diameter | |
| 875 | 20 | M | 10-Dec-96 | 18-Sep-97 | 2 | Itching, scaling, redness, | 95% of regression of lesions after 1 |
| | | | | | | edema, since 5 months ago | dose |
| | | | | | | in head, trunk, arms and legs | |
| 905 | 16 | F | 28-Jan-97 | 13-Mar-97 | 3 | Itching, bleeding, scaling, | 25-50% regression of lesions after 2 |
| | | | | | | redness, head, trunk, arms, | doses |
| | | | | | | legs | |
| 1071 | 10 | M | 7-Mar-97 | 26-Feb-98 | 8 | Itching, wounds, bleeding, | 80-90% regression of lesions after 1 |
| | | | | | | erythema, scaling in neck, | dose, moderate relapse after 2 doses, |
| | | | | | | knees, elbows, axils, | regression of lesions starts again after |
| | | | | | | | 3 doses and is 80-90% after 4 doses |
| | | | | | | | continuing thru 8 doses |
| 1473 | 16 | F | 4-Aug-97 | 18-Sep-97 | 2 | Redness, plaques, scaling | 100% clinical remission of lesions |
| | | | | | | on head and neck started 1 | after 1 dose |
| | | | | | | year ago | |
| 1727 | 35 | F | 28-Oct-97 | 1-Apr-98 | 6 | Redness, plaques and | 50% regression of lesions after 1 |
| | | | | | | scaling in trunk, arms, legs, | dose for 10 days, after 2 doses 50% |
| | | | | | | and head | regression of lesions for 4 days. |
| | | | | | | | Relapse after 3 doses, 70% |
| | | | | | | | regression of lesions for 25 days after |
| | | | | | | | 5 doses, after 6 doses 50% |
| | | | | | | | regression of lesions |
| 1906 | 14 | F | 3-Feb-98 | 30-Apr-98 | 5 | Itching, erythema, scaling, | 50% of regression of lesions after 1 |
| | | | | | | wounds, on entire body | dose, 90% regression after 2 doses, |
| | | | | | | | 100% regression after 3 doses |
| | | | | | | | maintaining after 4 doses |
| 1937 | 4 | M | 11-Feb-98 | 11-Jun-98 | 4 | Itching, wounds, scaling, | After 1 dose, 90% regression on face, |
| | | | | | | bleeding, redness in trunk, | 10% regression on arms and legs, 75% |
| | | | | | | arms, legs, face | regression on trunk. Arms and |
| | | | | | | | Legs continue with 50% regression |
| | | | | | | | after 2 doses. After 3 doses 90% |
| | | | | | | | regression of all lesions |
| 1944 | 7 | F | 12-Feb-98 | 2-Sep-98 | 3 | Redness, itching, scaling all | 50% regression after 1 dose |
| | | | | | | over | continuing at 50% after 2 doses |

S200 Amastigotes Peptides Homology with Different Proteins:
AS200 vaccine had 114 peptide sequences, showing 100% homology with several proteins. Peptides sequences at ≧3.5% proportions in relation to total peptides with more probability of interacting with the immunological synapses and peptides <3.5% proportion, with less probability of interacting with T and APC are shown in Table 1. Cationic peptides frequency in AS200 amastigotes vaccine:
Peptide charge was calculated from each amino acid present, at pH 7.4 (Hausman and Cooper 2004, The cell: a molecular approach. Washington, D.C. ASM Press. p. 51. ISBN 0-87893-214-3) and peptides hydropathy from published amino acids values (Kyte and Doolittle 1982, J. Mol. Biol. 157:105-32). Uncharged peptides were found at 31% frequency; cationic peptides at 20%, more abundant in antithrombin III, major surface GP63, α-tubulin, tubulin-β chain and putative mitochondrial peroxiredoxin (Table 1).

### Anionic Peptides Frequency in AS200 Amastigotes Vaccine:

Anionic peptides found in 49% of peptides predominated in the amastigotes VT vaccine, more abundant in the following proteins: antithrombin III, Hsp83, serine proteinase inhibitor, elongation factor 1-α, Glutamate dehydrogenase precursor, Serum albumin precursor, α-tubulin, tubulin-β chain, putative mitochondrial peroxiredoxin, Cysteine proteinase B precursor, IgM heavy chain constant, Alpha 2HS glycoprotein, peroxidoxin 1, Heat shock protein 70, Polyubiquitin 5, and ADP-ribosylation factor (Table 1). These peptides could bind more frequently the synaptic complex, sensitizing T cells, since no antibodies were found in patients with psoriasis after 6 doses of VT AS100-1, in comparison to patients with active leishmaniasis used as positive controls (O'Daly et al. 2009, Arch. Dermatol. Res. 301:1-13). Stimulation of cellular immunity measured by DTH in vivo and blastogenesis in vitro was found with amastigotes peptides vaccines (O'Daly et al. 2009, Arch. Dermatol. Res. 301:1-13. O'Daly et al. 2009, Arch. Dermatol. Res. 301:411-427. O'Daly et al. 2010, Arch. Dermatol. Res. 302:567-581). Several anionic peptides had low hydropathy values and high negative charges increasing the probability of interaction with the MHC grove and the TCR active site.

### Example 8

A field trial for Seborrheic Dermatitis in 6 volunteers, 66% females average age 32.8±7.9 years old range 23-40 years old, with 3.8±1.9 doses VT AS100-1 polyvalent vaccine also had a positive benefit/risk ratio in 6 years of follow up (Table 5).

TABLE 5
SEBORRHEIC DERMATITIS

| | | | | | # of | | |
|---|---|---|---|---|---|---|---|
| | | | Treatment | Treatment | Doses | | Description of Clinical Effect After |
| Case # | Age | Sex | Start Date | End Date | Total | Baseline Diagnosis | Treatment |
| 235 | 40 | F | 6-Jan-95 | 17-May-95 | 5 | Abundant dandruff in scalp, | After 1 dose no itching no dandruff, |
| | | | | | | itching, | after 2 doses 100% remission, after 3 |
| | | | | | | | doses some dandruff, after 4 doses |
| | | | | | | | relapse red areas in scalp and scaling |
| 345 | 23 | F | 5-Jun-95 | 8-Oct-95 | 4 | Started one year ago. Scaling | After 1 dose less itching, after 2 doses |
| | | | | | | plaques in scalp, and eyebrow. | 50% less lesions. Relapse one month |
| | | | | | | Itching | ago only scaling no itching |
| 763 | 24 | F | 28-Aug-96 | 1-Oct-96 | 2 | Itching, red areas, | After 1 dose no bleeding, moderate |
| | | | | | | hiperqueratosis in follicle, | itching and redness, scaling increased |
| | | | | | | scaling, bleeding In scalp | |
| | | | | | | since 5 years ago. | |
| 962 | 40 | F | 13-Mar-97 | 21-May-97 | 3 | Pain, itching, scaling, redness | After 1 dose 50% regression of lesions, |
| | | | | | | one year ago in head | after 2 doses no scaling, no erythema |
| | | | | | | | and 75% regression of lesions |
| 991 | 31 | M | 7-Apr-97 | 8-May-97 | 2 | Scaling and redness in ears, | 15 days after 1 dose remission of |
| | | | | | | scalp and eyebrows. | lesions then relapse |
| 1329 | 39 | M | 17-Jul-97 | 29-Jul-98 | 7 | Itching, scaling, redness, | 100% remission of lesions after 1 dose |
| | | | | | | started 8 years ago in scalp | continuing through 6 doses |

### Example 9

The foregoing description of specific embodiments is merely illustrative, and various modifications may be made without deviating from the spirit and scope of the present invention, which is limited only by the following claims.

What is claimed is:

1. An immunotherapeutic agent, consisting All 114 peptides sequences and corresponding proteins as listed in Table 3 capable of eliciting an immune response to result in abatement of the clinical symptoms and signs of cutaneous leishmaniasis, psoriasis, psoriatic arthritis, rheumatoid arthritis, atopic dermatitis, seborrheic dermatitis and skin papilloma said agent comprising a purified protein extract wherein said purified extract is isolated by diethylaminoethyl Sephadex chromatography of a Nonidet P-40 insoluble particulate antigen fraction derived from isolated killed cells of amastigotes from at least one species of the Leishmania genus, said particulate antigen fraction solubilized with 8 M urea and 0.025 M. Tris[hydroxymethyl]-aminomethane pH 8.3 applied to diethylaminoethyl Sephadex and eluted with a solution comprising 0.1 M. sodium chloride, 8 M urea and 0.025 M. Tris[hydroxymethyl]aminomethane pH 8.3, said purified protein extract including polypeptides having apparent molecular weights after total reduction and alkylation of 50 to 70 kDa.

2. The immunotherapeutic agent of claim 1 wherein the species is Leishmania amazonensis.

3. The immunotherapeutic agent of claim 1, wherein the species is Leishmania venezuelensis.

4. The immunotherapeutic agent of claim 1, wherein the species is Leishmania brasiliensis.

5. The immunotherapeutic agent of claim 1, wherein the species is Leishmania chagasi.

6. The immunotherapeutic agent of claim 1, wherein the species are Leishmania amazonensis, Leishmania venezuelensis, Leishmania brasiliensis and Leishmania chagasi.

7. The immunotherapeutic agent of claim 1 wherein a polypeptide comprising an isolated amino acid sequence or immunogenic variants thereof, the isolated amino acid sequence selected from the group of 114 peptides listed in Table 3.

8. The immunotherapeutic agent of any one of claims 1-7 further comprising an adjuvant.

9. The immunotherapeutic agent of claim 8, wherein the adjuvant is alumina.

10. An immunotherapeutic agent, capable of eliciting an immune response to result in abatement of the clinical symptoms and signs of cutaneous leishmaniasis, psoriasis, psoriatic arthritis, rheumatoid arthritis, atopic dermatitis, seborrheic dermatitis and skin papilloma said agent comprising a purified protein extract wherein said purified extract is isolated by diethylaminoethyl Sephadex chromatography of a Nonidet P-40 insoluble particulate antigen fraction derived from isolated killed cells of amastigotes from at least one species of the Leishmania genus, said particulate antigen fraction solubilized with 8 M urea and 0.025 M. Tris[hydroxymethyl]aminomethane pH 8.3 applied to diethylaminoethyl Sephadex and eluted with a solution comprising 0.15 M. sodium chloride, 8 M urea and 0.025 M. Tris[hydroxymethyl]aminomethane pH 8.3, said purified protein extract including polypeptide having apparent molecular weights after total reduction and alkylation of 50 to 70 kDa.

11. The immunotherapeutic agent of claim 10 wherein the species is Leishmania amazonensis.

12. The immunotherapeutic agent of claim 10, wherein the species is Leishmania venezuelensis.

13. The immunotherapeutic agent of claim 10, wherein the species is Leishmania brasiliensis.

14. The immunotherapeutic agent of claim 10, wherein the species is Leishmania chagasi.

15. The immunotherapeutic agent of claim 10, wherein the species are Leishmania amazonensis, Leishmania venezuelensis, Leishmania brasiliensis and Leishmania chagasi

16. The immunotherapeutic agent of claim 10 wherein a polypeptide comprising an isolated amino acid sequence or immunogenic variants thereof, the isolated amino acid sequence selected from the group of 114 peptides listed in Table 3.

17. The immunotherapeutic agent of any one of claims 10-16 further comprising an adjuvant.

18. The immunotherapeutic agent of claim 17, wherein the adjuvant is alumina.
